(19) United States
(12) Patent Application Publication
(10) Pub. No.: US 2023/0310799 A1
(43) Pub. Date: Oct. 5, 2023

(54) SURGICAL SYSTEMS INCLUDING FLEXIBLE MRI-COMPATIBLE SURGICAL CATHETERS FOR TRANSFERRING A SUBSTANCE TO AND/OR FROM A BRAIN OF A PATIENT

(71) Applicant: ClearPoint Neuro, Inc., Solana Beach, CA (US)

(72) Inventor: Peter G. Piferi, Orange, CA (US)

(21) Appl. No.: 18/175,638

(22) Filed: Feb. 28, 2023

Related U.S. Application Data
(60) Provisional application No. 63/324,720, filed on Mar. 29, 2022.

(51) Int. Cl. A61M 25/00

(52) U.S. Cl. CPC A61M 25/0068; A61M 25/0054; A61M 25/0023

(57) ABSTRACT

Surgical catheters with sufficient flexibility to deflect in response to brain shift in vivo while remaining in a target trajectory to a target in the brain have an elongate body and an exposed internal distal tip. The elongate bodies can be 0.5-10 feet long and the exposed distal tip can be provided as a tip end of a fused silica transfer tube that extends through the elongate body. The surgical catheters can be affixed to a skull using a bolt with a through channel and a bolt nut with a through channel. A distal end of the surgical catheters can extend through a guide sheath coupled to the bolt to reach the target.

### RELATED APPLICATIONS

This patent application claims the benefit of and priority to U.S. Provisional Ser. No. 63/324,720, filed Mar. 29, 2022, the contents of which are hereby incorporated by reference as if recited in full herein.

### FIELD OF THE INVENTION

The present invention relates to image-guided diagnostic or interventional systems that may be particularly suitable for providing therapies in the brain.

### BACKGROUND OF THE INVENTION

Various therapeutic and diagnostic procedures require that a substance be delivered (e.g., dispensed or infused) into a prescribed region of a patient, such as into a target deep brain location in the patient's brain, using a delivery catheter or cannula. It is often important or critical that the substance be delivered with high accuracy to the target region in the patient and without undue trauma to the patient.

In the past, a rigid cannula has been used with a surgical navigation frame attached to a skull of a patient defining a rigid coupling that extends into the brain. See, U.S. Pat. No. 10,105,485 and pending U.S. patent application Ser. No. 17/021,773, the contents of which are hereby incorporated by reference as if recited in full herein. While the rigid cannula configuration provides a secure delivery path to target for the medical procedure, the patient must remain in a stationary position to avoid movement of the brain relative to the surgical navigation frame and the rigid cannula inside the brain.

There is a need for alternate therapy systems that can accommodate longer duration procedures in the brain and/or accommodate patient movement.

### SUMMARY

According to some embodiments, a catheter is provided that is configured to move with brain shift for transferring a substance to and/or from a brain of a patient.

At least some portion of the catheter can be devoid of rigid material such as ceramic. For example, a tip or distal end of the catheter may comprise a rigid or stiff ceramic material but at least an intermediate segment of the distal end portion can be sufficiently flexible to positionally shift in response to brain shift. The catheter can be MRI-compatible.

Embodiments of the invention are directed to an intrabrain catheter. The intrabrain catheter includes an elongate body having a length in a range of 0.5-10 feet, the elongate body comprising a transfer tube that extends a full length of the elongate body and that extends out a distal end portion thereof to define an exposed tip. The intrabrain catheter has a proximal end portion that is configured to be external to a patient. At least a segment of the intrabrain catheter has sufficient flexibility to be able to bend at least 30 degrees relative to an axially extending straight linear axis in an unloaded, normal orientation. The intrabrain catheter has a distal end portion with sufficient rigidity to maintain a straight linear orientation for insertion through a tubular guide of a trajectory frame. The distal end portion of the intrabrain catheter is configured to have sufficient flexibility to be able to deflect in response to a deflection force applied by brain tissue during brain shift associated with patient movement.

The elongate body can have an outer tube that is polymeric and that surrounds a length of the transfer tube. The outer tube can have a wall thickness that is greater than the transfer tube. The transfer tube can be indirectly coupled to the outer tube and is non-extendable relative to the outer tube.

A distal end portion of the outer tube can taper radially outward in an axial direction from the transfer tube to an outer diameter that is constant over a length of the outer tube between the proximal and distal ends. Optionally, the proximal end can terminate at a mhn. The outer tube can have a length that is less than a length of the elongate body and is in a range of 0.5-10 feet, more typically in a range of about 6-36 inches, such as 24-36 inches. The transfer tube can have a length that is longer than the outer tube.

The intrabrain catheter can further include a second tube that is attached to the transfer tube and that resides between the transfer tube and the outer tube. The second tube can be formed of the same material as the transfer tube and can terminate a distance outside the outer tube before the tip of the intrabrain catheter.

The second tube and the transfer tube can both be formed of fused silica.

At least a segment of the elongate body of the intrabrain catheter can be devoid of rigid material such as ceramic.

The intrabrain catheter can be MRI-compatible.

The proximal end portion of the elongate body can be coupled to a connector with an internal cavity surrounding an exposed sub-length of the transfer tube.

The elongate body can include a first polymeric outer tube coupled to a second polymeric outer tube via an adapter member. The first polymeric outer tube can extend longitudinally spaced apart from the second polymeric outer tube. The first polymeric outer tube can reside closer to the proximal end portion than the second polymeric outer tube and can have a greater outer diameter and wall thickness than the second polymeric outer tube.

The elongate body can be provided by a polymeric outer tube with a constant outer diameter extending between a proximal end to a segment merging into a tapered distal end segment.

The elongate body can have a polymeric outer tube that is directly attached to a second tube extending about the transfer tube along a sub-length of the elongate body and the second tube can be directly attached to the transfer tube.

Yet other embodiments are directed to a medical system that includes: an intrabrain catheter; a sheath assembly with a guide sheath having a proximal end and an opposing distal end and with a lumen extending therethrough. The proximal end has a shoulder that extends radially outward from the lumen. The medical system also includes a bolt configured to threadably engage a skull of a patient. The bolt has an open channel that extends axially therethrough. The guide sheath is configured to reside in the open channel of the bolt with the distal end residing distally of the bolt. The intrabrain catheter is configured to reside in the guide sheath with a distal end thereof residing external to the guide sheath. The medical system also includes a seal member inside the bolt adjacent the shoulder of the guide sheath and a bolt nut configured to couple to the bolt.

The proximal end of the sheath assembly can terminate inside the bolt.

The bolt nut can have a distal portion that is configured to apply a clamping force against the seal member.

The seal member can be an O-ring, optionally a silicone O-ring.

The intrabrain catheter can have an elongate body having a length in a range of 0.5-10 feet, such as in a range of about 3 feet to about 5 feet. The elongate body can have a transfer tube that extends a full length of the elongate body and that extends out a distal end portion thereof to define an exposed tip. The intrabrain catheter can have a proximal end portion that is configured to be external to a patient. The proximal end portion can have sufficient flexibility to be able to bend at least 30 degrees relative to an axially extending straight linear axis in an unloaded, normal orientation. The intrabrain catheter can have a distal end portion with sufficient rigidity to maintain a straight linear orientation for insertion through a tubular guide of a trajectory frame. The distal end portion of the intrabrain catheter can be configured to have sufficient flexibility to be able to deflect in concert with the guide sheath response to a deflection force applied by brain tissue during brain shift associated with patient movement.

Yet other embodiments are directed to methods of providing a therapy to a brain of a subject. The methods include: attaching a bolt with a through channel to a skull of the subject; inserting a guide sheath assembly into a trajectory guide, then into the through channel of the bolt; attaching the guide sheath assembly to the bolt with a guide sheath of the guide sheath assembly extending distally out of the through channel of the bolt into a brain of the subject; inserting a catheter into the trajectory guide with a distal end of the catheter extending outside of the guide sheath; and allowing the guide sheath and distal end portion of the catheter to deflect in response to brain shift.

The method can further include providing an insertion tool assembly with a stylet releasably attached to the guide sheath assembly and, before inserting the guide sheath assembly into the trajectory guide, slidably forcing the guide sheath assembly to couple to the bolt using the insertion tool assembly, then removing the insertion tool assembly before inserting the catheter into the trajectory guide.

The method can further include delivering a therapy to the brain using the catheter.

The catheter can have an elongate catheter body that is devoid of ceramic and has a length in a range of 0.5-5 feet and a maximum outer diameter in a range of 2 F-8 F.

According to some embodiments, a method of transferring a substance to and/or from a patient includes providing a catheter; inserting the catheter into a selected region in the patient; and transferring the substance to or from the selected region through the transfer tube.

In some embodiments, the method includes partially withdrawing the catheter from a patient tissue in the selected region, thereby forming a channel in the patient tissue; and delivering stem cells through the cannula into the channel.

In some embodiments, the selected region is the brain.

### BRIEF DESCRIPTION OF THE DRAWINGS

FIG. 1 is a schematic illustration of an image-guided surgical system according to some embodiments of the present invention.

FIG. 2 is a top or side view of an example catheter according to embodiments of the present invention.

FIG. 3 is an enlarged, side perspective, section view of a trajectory guide holding a catheter and forming a part of the image-guided surgical system of FIG. 1.

FIG. 4A is a schematic illustration of a catheter in the brain coupled to a guide sheath assembly according to embodiments of the present invention.

FIG. 4B is an enlarged sectional view of the catheter and guide sheath assembly shown in FIG. 4A.

FIG. 4C is a side perspective view of an example insertion tool assembly coupled to the guide sheath assembly shown in FIG. 4B (prior to engagement with the catheter) and aligned with a bolt according to embodiments of the present invention.

FIG. 4D is a side perspective view of the example insertion tool assembly shown in FIG. 4C with the guide sheath assembly inserted into and attached to the bolt so that the guide sheath extends below the bolt.

FIG. 5 is a fragmentary side view of a surgical catheter according to further embodiments of the present invention.

FIG. 6 is a cross-sectional view of the surgical catheter of FIG. 5 taken along the line 6-6 of FIG. 5.

FIG. 7 is an enlarged cross-sectional view of Detail 7 of FIG. 6.

FIG. 8 is an enlarged, cross-sectional view of Detail 8 of FIG. 6.

FIG. 9 is an enlarged, cross-sectional view of Detail 9 of FIG. 6.

FIG. 10 is a fragmentary, side view of a surgical catheter according to further embodiments of the present invention.

FIG. 11 is a cross-sectional view of the surgical catheter of FIG. 10, taken along line 11-11 in FIG. 10.

FIG. 12 is an enlarged cross-sectional view of Detail 12 of FIG. 11.

FIG. 13 is an enlarged, cross-sectional view of Detail 13 of FIG. 11.

FIG. 14 is an enlarged, cross-sectional view of Detail 14 of FIG. 11.

FIG. 15 is a data processing system according to some embodiments of the present invention.

FIG. 16 is a flow chart of example actions that can be used to provide a therapeutic treatment according to embodiments of the present invention.

### DETAILED DESCRIPTION

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. It will be appreciated that although discussed with respect to a certain embodiment, features or operation of one embodiment can apply to others.

In the drawings, the thickness of lines, layers, features, components and/or regions may be exaggerated for clarity and broken lines (such as those shown in circuit of flow diagrams) illustrate optional features or operations, unless specified otherwise. In addition, the sequence of operations (or steps) is not limited to the order presented in the claims unless specifically indicated otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when a feature, such as a layer, region or substrate, is referred to as being “on” another feature or element, it can be directly on the other element or intervening elements may also be present. In contrast, when an element is referred to as being “directly on” another feature or element, there are no intervening elements present. It will also be understood that, when a feature or element is referred to as being “connected” or “coupled” to another feature or element, it can be directly connected to the other element or intervening elements may be present. In contrast, when a feature or element is referred to as being “directly connected” or “directly coupled” to another element, there are no intervening elements present. Although described or shown with respect to one embodiment, the features so described or shown can apply to other embodiments.

The term “electroanatomical visualization” refers to a visualization or map of the anatomical structure, e.g., brain, typically a volumetric, 3-D map or 4-D map, that illustrates or shows electrical activity of tissue correlated to anatomical and/or coordinate spatial position. The visualization can be in color and color-coded to provide an easy-to-understand map or image with different measures or gradients of activity in different colors and/or intensities. The term “color-coded” means that certain features, electrical activity or other output are shown with defined regions of different color and/or intensity to visually accentuate different tissue, different and similar electrical activity or potential in tissue and/or to show abnormalities or lesions in tissue versus normal or non-lesion tissue. In some embodiments, the systems can be configured to allow a clinician to increase or decrease the intensity or change a color of certain tissue types or electrical outputs, e.g., in high-contrast color and/or intensity, darker opacity or the like.

The actual visualization can be shown on a screen or display so that the map and/or anatomical or tool structure is in a flat 2-D view and/or in 2-D what appears to be 3-D volumetric images with data representing features or electrical output with different visual characteristics such as with differing intensity, opacity, color, texture and the like. For example, the 3-D image of the lung can be generated to illustrate differences in barrier thickness using color or opacity differences over the image volume. Thus, the term “3-D” in relation to images does not require actual 3-D viewability (such as with 3-D glasses), just a 3-D appearance, typically on a display. The 3-D images comprise multiple 2-D slices. The 3-D images can be volume renderings well known to those of skill in the art and/or a series of 2-D slices, which can be visually paged through. A 4-D map illustrates time-dependent activity, such as electrical activity or blood flow movement.

The surgical systems may be configured to operate based on known physical characteristics of one or more surgical tools, which may include a surgical (e.g., delivery) catheter, bolt that attaches to a skull, fiducials on a trajectory guide, and/or other component(s), such that the hardware is a point of interface for the circuit or software. The systems can communicate with databases that define dimensions, configurations or shapes and spacing of components on the tool(s). The defined physical data can be obtained from a CAD model of a tool. The physical characteristics can include dimensions or other physical features or attributes and may also include relative changes in position of certain components or features upon a change in position of a tool or portion thereof. The defined physical characteristics can be electronically (programmatically) accessible by the system or known a priori and electronically stored locally or remotely and used to automatically calculate certain information and/or to segment image data. That is, tool data from the known dimensions and configuration of the tool model can be used to segment image data and/or correlate a position and orientation of a tool and/or provide trajectory adjustment guidelines or error estimates, warnings of improper trajectories and the like. For example, the system can include defined structural and/or operational details/data for one or more of a delivery catheter, a grid for marking a burr hole location and/or a trajectory guide. The system can use this data to allow a user to adjust an intrabrain path for placing a diagnostic or therapy device. Such can be input, transposed, and/or overlayed in a visualization of the tool on one or more displays along with patient structure or otherwise used, such as, for example, to project the information onto a patient's anatomical structure or determine certain operational parameters including which image volume (scan planes) to use to obtain image data that will include select portions of the targeting cannula or surgical catheter. The image-guided systems can be MRI-image guided systems. As such, at least some of the generated visualizations are not merely an MRI image of the patient during a procedure.

The visualizations are rendered visualizations that can combine multiple sources of data to provide visualizations of spatially encoded tool position and orientation with anatomical structure and can be used to provide position adjustment data output so that a clinician can obtain a desired trajectory path, thereby providing a smart-adjustment system without requiring undue “guess” work on what adjustments to make to obtain the desired trajectory.

The term “animation” refers to a sequence or series of images shown in succession, typically in relatively quick succession, such as in about 1-50 frames per second. The term “frame” refers to a single visualization or static image. The term “animation frame” refers to one image frame of the different images in the sequence of images.

The term “ACPC coordinate space” refers to a right-handed coordinate system defined by anterior and posterior commissures (AC, PC) and Mid-Sagittal plane points, with positive directions corresponding to a patient's anatomical Right, Anterior and Head directions with origin at the mid-commissure point.

The term “grid” refers to a pattern of crossed lines or shapes used as a reference for locating points or small spaces, e.g., a series of rows and intersecting columns, such as horizontal rows and vertical columns (but orientations other than vertical and horizontal can also be used). The grid can include associated visual indicia such as alphabetical markings (e.g., A-Z and the like) for rows and numbers for columns (e.g., 1-10) or the reverse. Other marking indicia may also be used. The grid can be provided as a flexible patch that can be releasably attached to the skull of a patient. For additional description of suitable grid devices, see co-pending, co-assigned U.S. patent application Ser. No. 12/236,621 (U.S. Published Patent Application No. US 2009/0177077 A1), the disclosure of which is incorporated herein by reference.

The term “fiducial marker” refers to a marker that can be electronically identified using image recognition and/or electronic interrogation of MRI image data. The fiducial marker can be provided in any suitable manner, such as, but not limited to, a geometric shape of a portion of the tool, a component on or in the tool, a coating or fluid-filled component or feature (or combinations of different types of fiducial markers) that makes the fiducial marker(s) MRI-visible with sufficient signal intensity (brightness) or generates a “void” or dark space for identifying location and/or orientation information for the tool and/or components thereof in space.

The term “MM scanner” refers to a magnetic resonance imaging and/or NMR spectroscopy system. As is well known, MRI scanners include a low field strength magnet (typically between about 0.1 T to about 0.5 T), a medium field strength magnet, or a high-field strength super-conducting magnet, an RF pulse excitation system, and a gradient field system. MM scanners are well known to those of skill in the art. Examples of commercially available clinical MRI scanners include, for example, those provided by General Electric Medical Systems, Siemens, Philips, Varian, Bruker, Marconi, Hitachi and Toshiba. The MRI systems can be any suitable magnetic field strength, such as, for example, about 1.5 T or about 3.0 T, and may include other high-magnetic field systems between about 2.0 T-10.0 T.

The term “RF safe” means that the lead or probe is configured to safely operate when exposed to RF signals, particularly RF signals associated with MRI systems, without inducing unplanned current that inadvertently unduly heats local tissue or interferes with the planned therapy.

The term “MRI visible” means that the device is visible, directly or indirectly, in an MM image. The visibility may be indicated by the increased SNR of the MRI signal proximate the device.

The term “MM compatible” means that the so-called component(s) is suitable for use in an MRI environment and as such is typically made of a non-ferromagnetic MM compatible material(s) suitable to reside and/or operate in or proximate a conventional medical high magnetic field environment. The “MM compatible” component or device is “MR safe” when used in the MRI environment and has been demonstrated to neither significantly affect the quality of the diagnostic information nor have its operations affected by the MR system at the intended use position in an MR system. These components or devices may meet the standards defined by ASTM F2503-05. See, American Society for Testing and Materials (ASTM) International, Designation: F2503-05. Standard Practice for Marking Medical Devices and Other Items for Safety in the Magnetic Resonance Environment. ASTM International, West Conshohocken, PA, 2005.

The term “near real time” refers to both low latency and high frame rate. Latency is generally measured as the time from when an event occurs to display of the event (total processing time). For tracking, the frame rate can range from between about 100 fps to the imaging frame rate. In some embodiments, the tracking is updated at the imaging frame rate. For near ‘real-time’ imaging, the frame rate is typically between about 1 fps to about 20 fps, and in some embodiments, between about 3 fps to about 7 fps. The low latency required to be considered “near real time” is generally less than or equal to about 1 second. In some embodiments, the latency for tracking information is about 0.01 s, and typically between about 0.25-0.5 s when interleaved with imaging data. Thus, with respect to tracking, visualizations with the location, orientation and/or configuration of a known intrabody device can be updated with low latency between about 1 fps to about 100 fps. With respect to imaging, visualizations using near real time MR image data can be presented with a low latency, typically within between about 0.01 ms to less than about 1 second, and with a frame rate that is typically between about 1-20 fps. Together, the system can use the tracking signal and image signal data to dynamically present anatomy and one or more intrabody devices in the visualization in near real-time. In some embodiments, the tracking signal data is obtained and the associated spatial coordinates are determined while the MR image data is obtained and the resultant visualization(s) with the intrabody device (e.g., surgical cannula) and the near RT MR image(s) are generated.

The term “automatically” means that the operation can be substantially, and typically entirely, carried out without human or manual input, and is typically programmatically directed or carried out. The term “electronically” includes both wireless and wired connections between components. The term “programmatically” means under the direction of a computer program that communicates with electronic circuits and other hardware and/or software.

The term “surgical catheter” refers to an intrabody catheter used to transfer a substance to and/or from a target intrabody location.

Embodiments of the invention may be particularly suitable for use with human patients but may also be used with any animal or other mammalian subject.

Embodiments of the present invention may take the form of an entirely software embodiment or an embodiment combining software and hardware aspects, all generally referred to herein as a “circuit” or “module.” In some embodiments, the circuits include both software and hardware and the software is configured to work with specific hardware with known physical attributes and/or configurations. Furthermore, the present invention may take the form of a computer program product on a computer-usable storage medium having computer-usable program code embodied in the medium. Any suitable computer readable medium may be utilized including hard disks, CD-ROMs, optical storage devices, a transmission media such as those supporting the Internet or an intranet, or other storage devices.

Computer program code for carrying out operations of the present invention may be written in an object-oriented programming language such as Java®, Smalltalk or C++. However, the computer program code for carrying out operations of the present invention may also be written in conventional procedural programming languages, such as the “C” programming language. The program code may execute entirely on the user's computer, partly on the user's computer, as a stand-alone software package, partly on the user's computer and partly on another computer, local and/or remote or entirely on the other local or remote computer. In the latter scenario, the other local or remote computer may be connected to the user's computer through a local area network (LAN) or a wide area network (WAN), or the connection may be made to an external computer (for example, through the Internet using an Internet Service Provider).

Embodiments are described in part below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams of certain of the figures herein illustrate exemplary architecture, functionality, and operation of possible implementations of embodiments of the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order or two or more blocks may be combined, depending upon the functionality involved.

Generally stated, some embodiments of the present invention are directed to MRI-guided systems that can generate substantially real time (e.g., near real-time) patient-specific visualizations of the patient and one or more surgical tools, including an MM-compatible intrabody surgical catheter (e.g., delivery catheter) and the delivery distribution, location, pattern, etc., in logical space and provide feedback to a clinician to improve the speed and/or reliability of an intrabody infusion or delivery of a substance to a target within the body through the delivery catheter.

Some embodiments of the present invention are directed to MM-guided systems that can generate substantially real time patient-specific visualizations of the patient and a distribution of a substance delivered to a target within the patient through an MRI-compatible delivery catheter in logical space and provide feedback to a clinician to improve the speed and/or reliability of an intrabody infusion or delivery of the substance. These systems can show a dynamic dispersion and/or infusion pattern of the substance delivered or dispensed into the patient. MRI can be effectively used to monitor the efficacy and/or delivery of the substance from the catheter.

In some embodiments, the image-guided system can be configured to interrogate and segment image data to locate fiducial markers in the image (e.g., an increased higher intensity pixel/voxel region and/or void created in the MRI image by the presence of the delivery cannula in the patient's tissue) and generate successive visualizations of the patient's anatomical structure and the delivery cannula using MRI image data and a priori data of the delivery cannula to provide (substantially real-time) visualizations of the distribution of the substance in the patient.

Some embodiments of the present invention can provide visualizations to allow more precise control, delivery, and/or feedback of an infusion or delivery therapy so that the therapy or delivery catheter associated therewith can be more precisely placed and/or so that the catheter or delivery can be adjusted to provide the desired distribution in tissue, or to confirm proper delivery and allow near real-time visualization of the procedure.

Some embodiments of the present invention are directed to MM-compatible intrabody flexible delivery catheters that may be used to precisely deliver any suitable and desired substance (e.g., cellular, biological, and/or drug therapeutics) to the desired anatomy target. The delivery catheters can be used in and the systems can be configured to guide and/or place the delivery catheter in any desired internal region of the body of the patient, but may be particularly suitable for neurosurgeries and delivery of a substance to a target area or region within the brain. The delivery catheters and systems can be used for a fluid therapy delivery, optionally a gene and/or stem-cell based therapy delivery or other neural therapy delivery and allow user-defined custom targets in the brain or to other locations. Catheters, systems and methods of the invention may be used to treat patients by delivery of cellular/biological therapeutics into the desired anatomy to modify their cellular function. The cells (e.g., stem cells) may improve function.

The target region may be any suitable region or area within the patient body. According to some embodiments, the target region is a STN anatomical region, which may be identified and located with reference to standard anatomical landmarks. According to some embodiments, the target area is deep brain tissue such as a tumor or other undesirable tissue mass.

According to some embodiments, the target is intrathecal. The intrathecal target may be in the brain or spinal cord.

The substance delivered to the target region through the delivery catheter may be any suitable and desired substance. According to some embodiments, the substance is a liquid or slurry. In the case of a tumor, the substance may be a chemotherapeutic (cytotoxic) fluid. In some embodiments, the substance can include certain types of advantageous cells that act as vaccines or other medicaments (for example, antigen presenting cells such as dendritic cells). The dendritic cells may be pulsed with one or more antigens and/or with RNA encoding one or more antigen. Exemplary antigens are tumor-specific or pathogen-specific antigens. Examples of tumor-specific antigens include, but are not limited to, antigens from tumors such as renal cell tumors, melanoma, leukemia, myeloma, breast cancer, prostate cancer, ovarian cancer, lung cancer and bladder cancer. Examples of pathogen-specific antigens include, but are not limited to, antigens specific for HIV or HCV. In some embodiments, the substance may comprise radioactive material such as radioactive seeds. Substances delivered to a target area may include, but are not limited to, the following as shown in Table 1:

TABLE 1

| DRUG (generic name) | DISORDER(S) |
|---|---|
| caprylidene | Alzheimer's disease |
| donepezil | Alzheimer's disease |
| galantamine | Alzheimer's disease |
| memantine | Alzheimer's disease |
| Tacrine | Alzheimer's disease |
| vitamin E | Alzheimer's disease |
| ergoloid mesylates | Alzheimer's disease |
| riluzole | Amyotrophic lateral sclerosis |
| metoprolol | Benign essential tremors |
| primidone | Benign essential tremors |
| propanolol | Benign essential tremors |
| gabapentin | Benign essential tremors & Epilepsy |
| nadolol | Benign essential tremors & Parkinson's disease |
| zonisamide | Benign essential tremors & Parkinson's disease |
| carmustine | Brain tumor |
| lomustine | Brain tumor |
| methotrexate | Brain tumor |
| cisplatin | Brain tumor & Neuroblastoma |
| ioversol | Cerebral arteriography |
| mannitol | Cerebral Edema |
| dexamethasone | Cerebral Edema & Neurosarcoidosis |
| baclofen | Cerebral spasticity |
| ticlopidine | Cerebral thrombosis/embolism |
| isoxsuprine | Cerebrovascular insufficiency |
| cefotaxime | CNS infection & Meningitis |
| acyclovir | Encephalitis |
| foscarnet | Encephalitis |
| ganciclovir | Encephalitis |
| interferon alpha-2a | Encephalitis |
| carbamazepine | Epilepsy |
| clonazepam | Epilepsy |
| diazepam | Epilepsy |
| divalproex sodium | Epilepsy |
| ethosuximide | Epilepsy |
| ethotoin | Epilepsy |
| felbamate | Epilepsy |
| fosphenytoin | Epilepsy |
| levetiracetam | Epilepsy |
| mephobarbital | Epilepsy |
| paramethadione | Epilepsy |
| phenytoin | Epilepsy |
| trimethadione | Epilepsy |
| pregabalin | Epilepsy & Neuralgia |
| immune globulin intravenous | Guillain-Barre Syndrome |
| interferon beta-1b | Guillain-Barre Syndrome & Multiple sclerosis |
| azathioprine | Guillain-Barre Syndrome & Multiple sclerosis & |
| | Neurosarcoidosis |
| risperidone | Head injury |
| tetrabenazine | Huntington's disease |
| acetazolamide | Hydrocephalus & Epilepsy |
| alteplase | Ischemic stroke |
| clopidogrel | Ischemic stroke |
| nimodipine | Ischemic stroke & Subarachnoid hemorrhage |
| Aspirin | Ischemic stroke & Thromboembolic stroke |
| amikacin | Encaphalitis |
| ampicillin | Encaphalitis |
| ampicillin/sulbactam | Encaphalitis |
| ceftazidime | Encaphalitis |
| ceftizoxime | Encaphalitis |
| cefuroxime | Encaphalitis |
| chloramphenicol | Encaphalitis |
| cilastatin/imipenem | Encaphalitis |
| gentamicin | Encaphalitis |
| meropenem | Encaphalitis |
| metronidazole | Encaphalitis |
| nafcillin | Encaphalitis |
| oxacillin | Encaphalitis |
| piperacillin | Encaphalitis |
| rifampin | Encaphalitis |
| sulfamethoxazole/trimethoprim | Encaphalitis |
| tobramycin | Encaphalitis |
| triamcinolone | Encaphalitis |
| vancomycin | Encaphalitis |
| ceftriaxone | Encaphalitis & Neurosyphilis |
| pennicillin | Encaphalitis & Neurosyphilis |
| corticotropin | Multiple sclerosis |
| dalfampridine | Multiple sclerosis |
| glatiramer | Multiple sclerosis |
| mitoxantrone | Multiple sclerosis |
| natalizumab | Multiple sclerosis |
| modafinil | Multiple sclerosis |
| cyclophosphamide | Multiple sclerosis & Brain tumor & Neuroblastoma |
| interferon beta-la | Multiple sclerosis & Neuritis |
| prednisolone | Multiple sclerosis & Neurosarcoidosis |
| prednisone | Multiple sclerosis & Neurosarcoidosis |
| amantadine | Multiple sclerosis & Parkinson's disease |
| methylprednisolone | Neuralgia |
| desvenlafaxine | Neuralgia |
| nortriptyline | Neuralgia |
| doxorubicin | Neuroblastoma |
| vincristine | Neuroblastoma |
| albendazole | Neurocystecercosis |
| chloroquine phosphate | Neurosarcoidosis |
| hydroxychloroquine | Neurosarcoidosis |
| infliximab | Neurosarcoidosis |
| pentoxyfilline | Neurosarcoidosis |
| thalidomide | Neurosarcoidosis |
| apomorphine | Parkinson's disease |
| belladonna | Parkinson's disease |
| benztropine | Parkinson's disease |
| biperiden | Parkinson's disease |
| bromocriptine | Parkinson's disease |
| carbidopa | Parkinson's disease |
| carbidopa/entacapone/levodopa | Parkinson's disease |
| carbidopa/levodopa | Parkinson's disease |
| entacapone | Parkinson's disease |
| levodopa | Parkinson's disease |
| pergolide mesylate | Parkinson's disease |
| pramipexole | Parkinson's disease |
| procyclidine | Parkinson's disease |
| rasagiline | Parkinson's disease |
| ropinirole | Parkinson's disease |
| rotiotine | Parkinson's disease |
| scopolamine | Parkinson's disease |
| tolcapone | Parkinson's disease |
| trihexyphenidyl | Parkinson's disease |
| seleginline | Parkinson's disease |
| rivastigmine | Parkinson's disease & Alzheimer's disease |
| anisindione | Thromboembolic stroke |
| warfarin | Thromboembolic stroke |
| 5-hydroxytryptophan | Depression & Anxiety & ADHD |
| duloxetine | Depression & Anxiety & Bipolar disorder |
| escitalopram | Depression & Anxiety & Bipolar disorder |
| venlafaxine | Depression & Anxiety & Bipolar disorder & Autism & |
| | Social anxiety disorder |
| desvenlafaxine | Depression & Anxiety & PTSD & ADHD |
| paroxetine | Depression & Anxiety & PTSD & Social anxiety disorder |
| fluoxetine/olanzapine | Depression & Bipolar disorder |
| 1-methylfolate | Depression & BPD |
| amitriptyline | Depression & PTSD |
| sertraline | Depression & PTSD & Bipolar disorder & Social anxiety |
| | disorder |
| fluvoxamine | Depression & PTSD & Social anxiety disorder |
| olanzapine | Depression & Schizophrenia & Bipolar disorder |
| paliperidone | Depression & Schizophrenia & Bipolar disorder |
| aripiprazole | Depression & Schizophrenia & Bipolar disorder & Autism |
| quetiapine | Depression & Schizophrenia & PTSD & BPD & Bipolar |
| | disorder |
| | Depression & Schizophrenia & PTSD & BPD & Bipolar |
| risperidone | disorder & Autism |
| amisulpride | Depression & Social anxiety disorder |
| chlorpromazine | Psychosis |
| droperidol | Psychosis |
| fluphenazine | Psychosis |
| periciazine | Psychosis |
| perphenazine | Psychosis |
| thiothixene | Psychosis |
| triflupromazine | Psychosis |
| haloperidol | Psychosis & Dementia |
| prazosin | PTSD |
| clozapine | Schizophrenia |
| flupenthixol | Schizophrenia |
| iloperidone | Schizophrenia |
| loxapine | Schizophrenia |
| mesoridazine | Schizophrenia |
| promazine | Schizophrenia |
| reserpine | Schizophrenia |
| thioridazein | Schizophrenia |
| zuclopenthixol | Schizophrenia |
| asenapine | Schizophrenia & Bipolar disorder |
| levomepromazine | Schizophrenia & Bipolar disorder |
| ziprasidone | Schizophrenia & Bipolar disorder |
| molindone | Schizophrenia & Psychosis |
| pimozide | Schizophrenia & Psychosis |
| thioridazine | Schizophrenia & Psychosis |
| Cytarabine | Chemotherapy, hematological malignancies |

According to some embodiments, the surgical catheter is used to remove or withdraw a substance therethrough from the target area. According to some embodiments, the surgical catheter is used to remove cerebral spinal fluid from the patient.

Embodiments of the present invention may include steps, features, aspects, components, procedures and/or systems as disclosed in U.S. Published Patent Application No. 2009/0171184, and PCT Published Patent Application No. WO 2011/130107 A2, the disclosures of which are incorporated herein by reference.

According to some embodiments, the systems are configured to provide a substantially automated or semi-automated and relatively easy-to-use, image-guided system with defined workflow steps and interactive visualizations. In particular embodiments, the systems define and present workflow with discrete steps for finding target and entry point(s), guiding the alignment of the targeting cannula to a planned trajectory, monitoring the insertion of the delivery cannula, and adjusting the (X-Y) position in cases where the placement needs to be corrected. During steps where specific MR scans are used, the circuit or computer module can display data for scan plane center and angulation to be entered at the console. The workstation/circuit can passively or actively communicate with the MR scanner. The system can also be configured to use functional patient data (e.g., fiber tracks, fMRI and the like) to help plan or refine a target surgical site and/or access path.

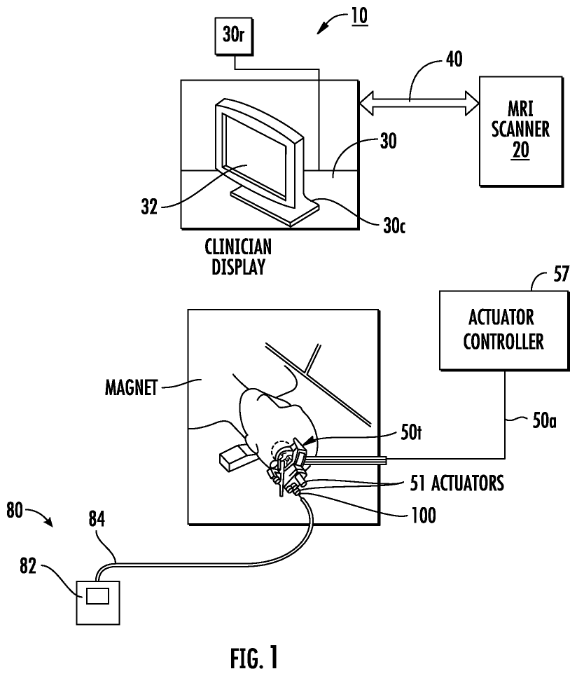

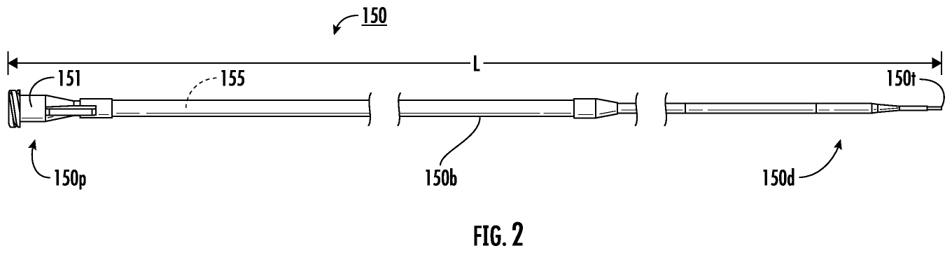

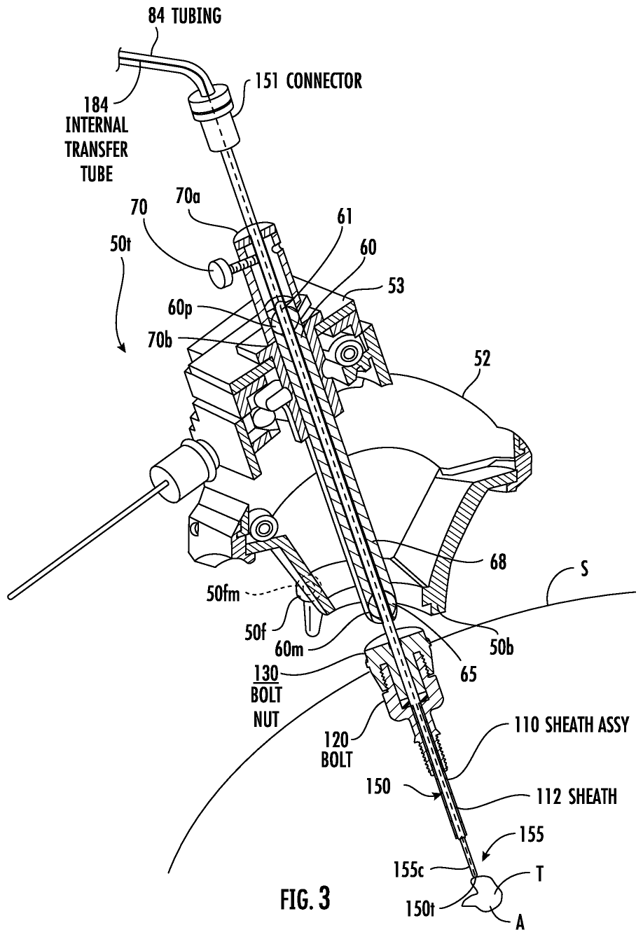

Embodiments of the present invention will now be described in further detail below with reference to the figures. FIG. 1 illustrates an image-guided interventional system 10 with an MRI scanner 20, a clinician workstation 30 with at least one circuit 30c, at least one display 32, a trajectory guide 50t, a depth stop 70 (FIG. 3), and a fluid substance transfer system 80. The fluid substance transfer system 80 includes a catheter 150 (FIG. 2) which may be an MRI-compatible intrabody surgical or delivery catheter. The fluid transfer system 80 can include an infusion or delivery pump 82 and connecting tubing 84.

The image-guided system 10 can be an MRI-guided system 10, although the image-guided system may be configured as a CT image guided system or be configured to work in both imaging modalities. The fluid transfer system 80 can be MM-compatible. The image-guided system 10 can be configured to render or generate real time visualizations of the target anatomical space using MRI image data and predefined data of at least one surgical tool to segment the image data and place the trajectory guide 50t and the catheter 150 in the rendered visualization in the correct orientation and position in 3D space, anatomically registered to a patient. The trajectory guide 50t and the catheter 150 can include or cooperate with tracking, monitoring and/or interventional components.

The tools of the system 10, including the catheter 150, can be provided as a sterile kit (typically as single-use disposable hardware) or in other groups or sub-groups or even individually, typically provided in suitable sterile packaging. The tools can also include a marking grid (e.g., as disclosed in U.S. Published Patent Application No. 2009/0177077 and/or U.S. Published Patent Application No. 2009/0171184). Certain components of the kit may be replaced or omitted depending on the desired procedure. Certain components can be provided in duplicate for bilateral procedures.

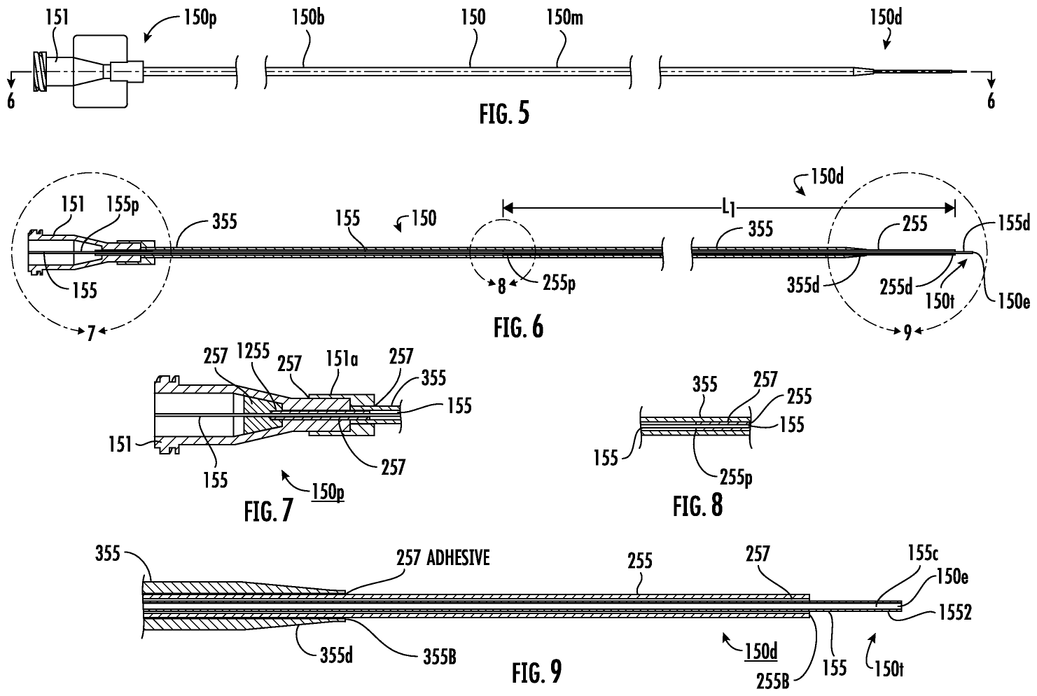

With reference to FIG. 2, an example catheter 150 is shown. The catheter 150 has a length L that is typically in a range of 0.5-5 feet, more typically about 45-51 inches. The catheter 150 is flexible about a segment, such as part of at least an intermediate segment between the distal end and the proximal end, such as part of distal end portion 150d upstream of the tip/distal end and has a catheter body 150b that surrounds a transfer tube 155 (FIGS. 6, 11), with the transfer tube 155 extending therethrough from a proximal end portion 150p with a connector 151 to a distal end portion 150d defining a tip 150t. The transfer tube 155 defines an open lumen or channel 155c (FIGS. 9, 14). The connector 151 is optional.

With reference to FIG. 3, the depth stop 70 has a generally cylindrical configuration with opposite proximal and distal ends 70a, 70b and is adapted to be removably secured within the proximal end of the tubular trajectory guide member 50t. The depth stop 70 can be attached to the catheter 150 to allow for a defined insertion depth where insertion depth control and/or locking, is desired.

An exemplary trajectory guide 50t is illustrated in FIGS. 1, 3 in position on a patient. As shown, the trajectory guide 50t (FIG. 3) includes a guide frame 50f, a targeting cannula 60 and trajectory guide actuators 51 having respective actuator cables 50c (FIG. 1) (providing X-Y adjustment and pitch and roll adjustment) in communication with a trajectory adjustment controller 57. The frame 50f can include a control arc 52 that cooperates with a platform 53 to provide pitch and roll adjustments. The platform 53 can allow for X-Y adjustments of the trajectory. The trajectory guide 50t may include a plurality of MRI-visible frame fiducial markers 50fm around a base 50b thereof. For additional discussion of suitable trajectory guides, see, U.S. Published Patent Application No. 2009/0112084 (Attorney Docket No. 9450-34IP), the contents of which are hereby incorporated by reference as if recited in full herein.

As shown in FIG. 3, the targeting cannula 60 includes an open center lumen or through passage 61 along the axis of the targeting cannula 60. The distal end portion of the targeting cannula 60 can include a fiducial marker 60m (typically including a fluid-filled component 65), shown as a substantially spherical or round (cross-section) marker shape. The proximal end 60p can be configured with a fluid filled channel 68 concentric with the passage 61 that can define a cylindrical fiducial marker. Other fiducial marker types can be used. The catheter 150 can be slidably introduced and/or withdrawn through the lumen or passage 61.

A scanner interface 40 (FIG. 1) may be used to allow communication between the workstation 30 and the scanner 20. The interface 40 and/or circuit 30c may be hardware, software or a combination of same. The interface 40 and/or circuit 30c may reside partially or totally in the scanner 20, partially or totally in the workstation 30, or partially or totally in a discrete device therebetween.

The scanner 20 can be an MRI scanner 20 can include a console that has a “launch” application or portal for allowing communication to the circuit 30c of the workstation 30. The scanner console can acquire volumetric T1-weighted (post-contrast scan) data or other image data (e.g., high resolution image data for a specific volume) of a patient's head or other anatomy. In some embodiments, the console can push DICOM images or other suitable image data to the workstation 30 and/or circuit 30c. The workstation 30 and/or circuit 30c can be configured to passively wait for data to be sent from the MR scanner 20 and the circuit 30c/workstation 30 does not query the scanner or initiate a communication to the scanner. In other embodiments, a dynamic or active communication protocol between the circuit 30c/workstation 30 and the scanner 20 may be used to acquire image data and initiate or request particular scans and/or scan volumes. Also, in some embodiments, pre-DICOM, but reconstructed image data, can be sent to the circuit 30c/workstation 30 for processing or display. In other embodiments, pre-reconstruction image data (e.g., substantially “raw” image data) can be sent to the circuit 30c/workstation 30 for Fourier Transform and reconstruction.

Referring to FIGS. 2 and 3, the catheter 150 can be configured to flowably introduce and/or inject a desired therapy A to a target site T (e.g., antigen, gene therapy, chemotherapy or stem-cell or other therapy type). The catheter 150 as shown in FIG. 3 includes a catheter body 150b with at least one longitudinally extending channel 155c extending from a first or inlet port in the connector 151 and at least one second or exit port at the tip 150t.

The catheter 150 can be formed of an MM-compatible material(s).

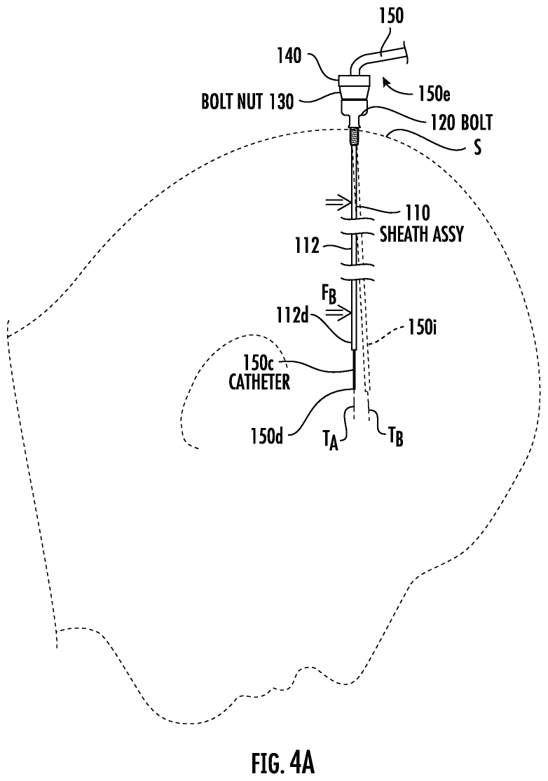

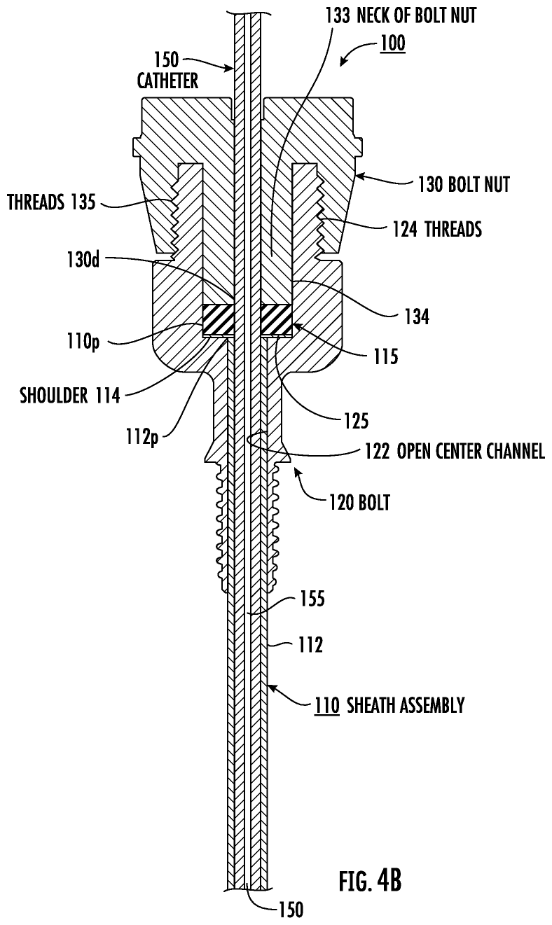

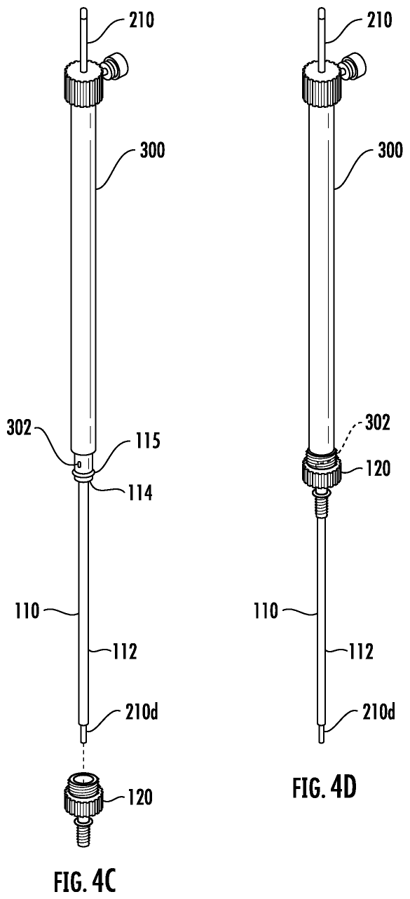

Referring to FIG. 4A, the catheter body 150b can have an external segment 150e that is sufficiently flexible to be able to bend at least 30 degrees from an orientation defined by an axially straight centerline without kinking or breaking. The catheter 150 can have an intrabody segment 150i that is sufficiently flexible to be able to deflect in response to a deflection load FB applied by tissue during brain shift. A segment or all of the catheter 150 can be devoid of rigid support material such as ceramic, at least outside the distal end portion 150d typically over an entire extent to the proximal end thereof.

Referring to FIGS. 3, 4A and 4B, the catheter 150 can be flexible with sufficient rigidity to be able to be self-supporting and retain a straight shape while being inserted through the trajectory guide 50t. The catheter 150 can be coupled to a bolt 120 that is pre-attached to the skull S of a patient. The bolt 120 secures a guide sheath assembly 110 in position. The catheter 150 can extend through a channel 122 in the bolt 120 and an aligned channel 134 in the bolt nut 130 and also extend through the guide sheath 112 of the guide sheath assembly 110. The catheter 150 can have a maximal outer diameter below the proximal end thereof that is in a range of 2F-8F, in some embodiments.

The proximal end 110p of the guide sheath assembly 110 can include a shoulder 114 that extends radially outward from the guide sheath 112. The bolt 120 has an open channel 122 that extends axially therethrough. A proximal portion 112p of the guide sheath 112 is configured to reside in the open channel 122 of the bolt 120 with the distal end 112d of the guide sheath 112 residing distally of the bolt 120. A seal member 115 can reside inside the bolt 120 adjacent the shoulder 114 of the guide sheath 112. The bolt nut 130 is configured to couple to the bolt 120 and secure the catheter 150 thereto to position the tip 150t of the catheter at a desired length beyond the guide sheath 112 at a target T.

The proximal end 110p of the sheath assembly 110 can terminate inside the bolt 120. The bolt nut 130 can have threads 135 which can engage threads 124 of the bolt 120. The threads 135, 124 can terminate above a distal portion 130d of the bolt nut 130. The bolt nut 130 can have a neck 133 that merges into the open channel 134. The distal portion 130d of the bolt nut 130 can be configured to apply a clamping force against the seal member 115. The seal member 115 can include or be defined by a silicone O-ring. The seal member 115 can inhibit liquid from flowing out of the body and into the channels 122, 134, for example.

As shown in FIG. 4A, a cap 140 can be coupled to the bolt nut 130 and provide a passage for a curvilinear portion of the external segment 150e of the catheter 150 to extend from the bolt channel 122 and the bolt nut channel 134.

The guide sheath 112 can have a length that is in a range of about 1 cm to about 12 cm. The guide sheath 112 can be configured to be cut to length at a location distal to the shoulder 114 and seal member 115. The length of the guide sheath 112 can be about 1 cm, about 1.5 cm, about 2 cm, about 2.5 cm, about 3 cm, about 3.5 cm, about 4 cm, about 4.5 cm, about 5 cm, about 5.5 cm, about 6 cm, about 6.5 cm, about 7 cm, about 7.5 cm, about 8 cm, about 8.5 cm, about 9 cm, about 9.5 cm, about 10 cm, about 10.5 cm, about 11 cm, about 11.5 cm, or about 12 cm.

The sheath assembly 110 can be provided as a set of sheath assemblies, each sheath assembly can have a sheath with a different length to thereby allow a user to select an appropriate sheath assembly with a guide sheath having a desired length to extend to a target site in the patient for a medical procedure.

The guide sheath 112 can have an outer diameter in a range of 2F to 8 F, with a wall thickness in a range of 0.002 inches to about 0.025 inches to thereby have a flexible body that can remain in position to a target and deflect (relative to a bolt affixed to the skull and/or the skull) in response to a deflection load applied by brain tissue during a brain positional shift when implanted. The deflection load can be small as can the positional movement of the guide sheath (and catheter held therein) such as in a range of 1 ounce to 3 ounces.

The guide sheath 112 can be formed of medical grade polymers or co-polymers such as, for example, polyethylene, polyimide, PEEK, PEBAX or TEFLON.

FIG. 4A schematically illustrates the flexibility of the catheter 150 which can be configured to be able to deflect/shift in response to a deflection load F_{B} applied by brain tissue during a brain positional shift between positions A and B (relative to the skull/bolt) in the brain while remaining implanted and defining a trajectory to the (shiftable) target according to embodiments of the present invention.

Referring to FIGS. 4A and 4B, the guide sheath 112 and the catheter 150 can be sufficiently flexible (alone and in combination) to be able to deflect relative to the skull S and/or bolt 120 in response to a deflection load or force F_{B} applied by tissue in the brain in response to brain shift that can occur upon patient movement. The deflection load or force F_{B} can be relatively small, such as in a range of 1 ounce to 3 ounces, in some embodiments. The guide sheath 112 and an implanted/intrabrain portion (e.g., the distal end portion 150d) of the catheter 150 can deflect with the target tissue T from a trajectory TA to trajectory TB relative to the skull S to provide a trajectory from the skull S and/or bolt 120 that aligns with the target tissue T. The deflection load F_{B} can be small as can the positional movement of the guide sheath 112 and catheter 150 held therein. The deflection can be in any direction responsive to brain shift movement of local tissue and can change over time, e.g., the implanted portions of the guide sheath 112 and catheter 150 can “float” or “shift” with brain shift as the patient moves. The deflection load F_{B} can be applied along an entire length or a sub-length of the intrabody/implanted portion of the guide sheath 112 and catheter 150.

At least the distal end portion 150d of the catheter 150 can be more flexible than the guide sheath 112. The catheter 150 and the guide sheath 112 can be sufficiently flexible, when coupled together, to be able to shift in concert in response to brain shift when implanted in the brain.

Referring to FIGS. 4C and 4D, an insertion tool assembly 300 with an elongate body that holds a stylet 210 is shown. The insertion tool assembly 300 is configured to place the guide sheath assembly 110 into the bolt 120 and the stylet 210 extends through the guide sheath 112 while the insertion tool assembly 300 extends through the trajectory guide 50t (FIG. 3) to place the guide sheath 112 at the desired trajectory path to target T (FIG. 3). The insertion tool assembly 300 with the stylet 210 is then removed and the catheter 150 inserted through the trajectory guide 50t to position the distal end portion 150d of the catheter 150 in/through the guide sheath in the body.

FIG. 4D is a side perspective view of the components shown in FIG. 4C with the guide sheath assembly 110 coupled to the bolt 120 and the guide sheath 112 extending below the bolt 120.

The stylet 210 can be inserted through an insertion tool assembly 300 and through the sheath assembly 110. The stylet 210 can be adjusted so that a distal end 210d extends out of a distal end 112d of the guide sheath 112. The shoulder 114 or the seal member 115 can contact the distal end 302 of the insertion tool assembly 300. A proximal end 210p of the stylet 210 can extend out of the insertion tool assembly 300.

The insertion tool assembly 300, coupled to the sheath assembly 110 to form a unit, as shown in FIG. 4C, can be inserted through the tower of the surgical navigation frame assembly 50t (FIG. 3). As shown in FIG. 4D, a portion of the stylet 210 extends into the bolt 120 and the distal end 302 of the insertion tool assembly 300 enters the bolt 120 to place the sheath assembly 110 into the bolt 120. A distal end 210d of the stylet 210 extends out of the guide sheath 112 and provides increased rigidity to allow proper intrabody positioning along the defined trajectory path to target T (FIG. 3). Once the sheath assembly 110 is in the bolt 120 with the shoulder 114 of the sheath assembly 110 in place in the bolt 120, placing the distal end portion 112d of the guide sheath 112 in the proper position, the insertion tool assembly 300 with the cooperating stylet 210 are slidably removed, typically together as a unit. The stylet 210 can be removed from guide sheath 112 (and the trajectory frame 50t) separately from the insertion tool assembly 300. In any event, the sheath assembly 110 remains in position in the bolt 120, with the shoulder 114 held directly or indirectly against a seat 125 inside the bolt 120 according to some embodiments, after the stylet 210 is slidably removed therefrom.

The bolt nut 130 (FIG. 4B) can then be threaded onto the bolt 120. The sheath assembly 110, the bolt 120 and the bolt nut 130 are coupled together in position to define at least part of a therapy delivery system. The bolt nut 130 is not typically fully tightened against the bolt 120 until the catheter 150 is inserted therethrough. For additional discussion of the insertion tool assembly and bolt assemblies, see co-pending U.S. Provisional Application Ser. No. 63/322,257, filed Mar. 22, 2022, the contents of which are hereby incorporated by reference as if recited in full herein.

As discussed above, the transfer tube 155 can be fluidly connected to a pump 82 by tubing 84. The tubing 84 may comprise flexible tubing. According to some embodiments, the tubing 84 is PVC tubing. According to some embodiments, the tubing 84 is silicone tubing. The tubing 84 can surround an internal transfer tube 184. The transfer tube 155 and the internal transfer tube 184 can be formed of a fused silica or other inert (MRI-compatible) material.

In some embodiments, the catheter 150 can be self-loading and not require tubing and/or a pump. See, e.g., co-pending U.S. Provisional Patent Application Ser. No. 62/950,521 (Attorney Docket 9450-129PR), the contents of which are hereby incorporated by reference as if recited in full herein.

According to some embodiments, the pump 82 is configured as a syringe (e.g., a hand syringe).

Generally described, for some unilateral scenarios, the user (e.g., doctor or surgeon) will proceed through a set of discrete workflow steps to load MR image data, identify a target point, identify an entry point, verify the planned trajectory, and align the targeting cannula 60. A target point or region can also be planned or refined based on real-time functional image data of a patient. The functional image data can include, but is not limited to, images of fiber tracks, images of activity in brain regions during vocalization (e.g., reading, singing, talking), or based on physical or olfactory or sense-based stimulation, such as exposure to electrical (discomfort/shock input), heat and/or cold, light or dark, visual images, pictures or movies, chemicals, scents, taste, and sounds or the like) and/or using fMRI or other imaging techniques. The enhanced visualization may give neurosurgeons a much clearer picture of the spatial relationship of a patient's brain structures. The visualizations can serve as a trajectory guide for delivering a substance to the body (e.g., to the brain) via the surgical (intrabody) catheter 150. In some embodiments, the visualizations can be generated using data collated from different types of brain-imaging methods, including conventional magnetic resonance imaging (MRI), functional MRI (fMRI), diffusion-tensor imaging (DTI) and even hyperpolarized noble gas MRI imaging. The MRI gives details on the anatomy, fMRI or other active stimulation-based imaging protocol can provide information on the activated areas of the brain, and DTI provides images of the network of nerve fibers connecting different brain areas. The fusion of one or all of these different images and the tool information can be used to produce a 3-D display with trajectory information that surgeons can manipulate.

Thus, a target location and trajectory can be planned, confirmed or refined based in-part on functional information of the patient. This functional information can be provided, in a user interface (UI) displayed on the display screen 32, in near real-time visualizations of the patient with the trajectory guide tools for trajectory path and target planning, e.g., visualize a patient's fiber track structures and/or functional information of a patient's brain for a surgeon's ease of reference. Knowing where susceptible or sensitive brain regions are or where critical fiber tracks are in the patient's brain, can allow a surgeon to plan a better or less-intrusive trajectory and/or allow a surgeon to more precisely reach a desired target site and/or more precisely place a device and/or deliver a planned therapy substance.

To align the targeting cannula 60, scan volumes can be defined by the system based on known dimensions of the cannula, such as a cannula length, a position of a proximal or distal marker on the cannula, and angulation and lateral (X-Y) pivot limit.

An estimated distance from the distal tip 150t of the catheter 150 and/or the distal end 112d of the guide sheath 112 to a reference point(s) on another component such as, for example, on the guide frame 50t (FIG. 3) or the targeting cannula 60 (e.g., the proximal end of the targeting cannula 60) and/or the bolt 120 can be determined and physically or visually marked on the catheter 150. The depth stop 70 can be secured about the catheter 150 at the marked location. The depth stop 70 can serve to limit the depth of insertion of the catheter 150 into the patient in a subsequent insertion step or steps.

The user can then (gradually) advance the guide sheath 112 and/or subsequently, the catheter 150, and acquire images (on the display of the UI) to verify the trajectory and/or avoid functionally sensitive structure as appropriate.

For some bilateral scenarios, the above steps can be repeated for both left and right sides, with the additional goal that the patient should not be moved into or out of the scanner. To satisfy that goal, trajectory planning should be completed for both sides prior to removing the patient from the scanner. Also, burring and frame attachment (the member that holds the trajectory guide to the patient's head) should be completed for both sides prior to moving the patient back into the scanner 20 to promote speed of the procedure.

The system 10 can be configured with a (hardware/software) interface that provides a network connection, e.g., a standard TCP/IP over Ethernet network connection, to provide access to MR scanner 20, such as the DICOM server. The workstation 30 can provide a DICOM C-STORE storage class provider. The scanner console can be configured to be able to push images to the workstation 30 and the workstation 30 can be configured to directly or indirectly receive DICOM MR image data pushed from an MR scanner console. Alternatively, as noted above, the system can be configured with an interface that allows for a dynamic and interactive communication with the scanner 20 and can obtain image data in other formats and stages (e.g., pre-DICOM reconstructed or raw image data).

As noted above, the system 10 can be configured so that hardware, e.g., the trajectory guide 50t constitutes a point of interface with the system (software or computer programs) because the circuit 30c is configured with predefined tool data that recognizes physical characteristics of specific tool hardware.

The system 10 may also include and implement a marking grid and/or non-uniformly spaced-apart frame fiducial markers as disclosed in U.S. patent application Ser. No. 12/236,854, published as U.S. Published Patent Application No. 2009/0171184, the contents of which are hereby incorporated by reference as if recited in full herein.

In some embodiments, circuit 30c can be configured so that the program application can have distinct ordered workflow steps that are organized into logical groups based on major divisions in the clinical workflow as shown in Table 2. A user may return to previous workflow steps if desired. Subsequent workflow steps may be non-interactive if requisite steps have not been completed. The major workflow groups and steps can include the following features or steps in the general workflow steps of “start”, “plan entry”, “plan target”, “navigate”, and “refine,” ultimately leading to delivering and visualizing the therapy (i.e., delivering the substance to the target through the catheter 150) as described in Table 2.

TABLE 2
Exemplary Clinical Workflow Groups/Steps

| Group | Step | Description |
|---|---|---|
| Start | Start | Set overall procedure parameters (Optionally |
| | | confirm hardware compatibility) |
| Plan Entry | ACPC | Acquire a volume and determine AC, PC, and |
| | | MSP points |
| | Target | Define initial target point(s) for entry planning |
| | Trajectory | Explore potential trajectories to determine entry |
| | | point(s) |
| | Grid | Locate physical entry point via fiducial grid. |
| Plan Target | ACPC | With hole burred and frame attached, acquire a |
| | | volume and determine revised AC, PC, and MSP |
| | | points. |
| | Target | Acquire high-resolution slabs (e.g., T2 slabs) to |
| | | determine target positions in new volume. |
| | Trajectory | Review final planned trajectory prior to starting |
| | | procedure. |
| Navigate | Initiate | Acquire slabs to locate initial position of |
| | | cannula. |
| | Alignment | Dynamically re-acquire scan showing position of |
| | | top of cannula. With each update show projected |
| | | target position to determine when alignment is |
| | | correct. |
| | Insertion | Acquire slabs as guide sheath 112 and/or |
| | | catheter 150 is inserted into brain. Verify that the |
| | | guide sheath 112 and/or catheter 150 is |
| | | following planned trajectory. |
| Refine | Target | Acquire images with the guide sheath 112 and/or |
| | | catheter 150 in place. Review position and |
| | | redefine target if necessary. |
| | Adjust XY | Dynamically re-acquire scan showing position of |
| | Offset | bottom of targeting cannula 60. With each |
| | | update show projected target position to |
| | | determine when offset is correct. |
| | Insertion | Acquire slabs as the guide sheath 112 and/or |
| | | catheter 150 is inserted into brain. Verify that |
| | | guide sheath 112 and/or catheter 150 is |
| | | following planned trajectory. |
| Delivery or | Substance Delivery | Once catheter 150 position is finalized, prompt |
| Withdrawal | | user to begin delivery or withdrawal of |
| | | substance through catheter 150. |
| | Watch Diffusion Delivery | Acquire slabs as substance is delivered into or |
| | Pattern | withdrawn from brain and display. |
| Admin | Admin | Reporting and Archive functionality |

The AC, PC and MSP locations can be identified in any suitable manner. In some embodiments, the AC-PC step can have an automatic, electronic AC, PC MSP Identification Library. The AC, PC and MSP anatomical landmarks define an AC-PC coordinate system, e.g., a Talairach-Tournoux coordination system that can be useful for surgical planning. This library can be used to automatically identify the location of the landmarks. It can be provided as a dynamic linked library that a host application can interface through a set of Application Programming Interface (API) on Microsoft Windows®. This library can receive a stack of MR brain images and fully automatically locates the AC, PC and MSP. The success rate and accuracy can be optimized, and typically it takes a few seconds for the processing. The output is returned as 3D coordinates for AC and PC, and a third point that defines the MSP. This library is purely computation and is typically UI-less. This library can fit a known brain atlas to the MR brain dataset. The utility can be available in form of a dynamic linked library that a host application can interface through a set of Application Programming Interface (API) on Microsoft Windows®. The input to this library can contain the MR brain dataset and can communicate with applications or other servers that include a brain atlas or include a brain atlas (e.g., have an integrated brain atlas). The design can be independent of any particular atlas; but one suitable atlas is the Cerefy® atlas of brain anatomy (note: typically not included in the library). The library can be configured to perform segmentation of the brain and identify certain landmarks. The atlas can then be fitted in 3D to the dataset based on piecewise affine transformation. The output can be a list of vertices of the interested structures.

In some embodiments, the mid-sagittal plane (MSP) is approximated using several extracted axial slices from the lower part of the input volume, e.g., about 15 equally spaced slices. A brightness equalization can be applied to each slice and an edge mask from each slice can be created using a Canny algorithm. A symmetry axis can be found for each edge mask and identify the actual symmetry axis based on an iterative review and ranking or scoring of tentative symmetry axes. The ranking/scoring cam be based on whether a point on the Canny mask, reflected through the symmetry axis lands on the Canny mask (if so, this axis is scored for that slice). An active appearance model (AAM) can be applied to a brain stem in a reformatted input stack with the defined MSP to identify the AC and PC points.

The MSP plane estimate can be refined as well as the AC and PC points. The MSP plane estimate can be refined using a cropped image with a small region that surrounds a portion of the brain ventricle and an edge mask using a Canny algorithm. The symmetry axis on this edge mask if found following the procedure described above. The AC and PC points are estimated as noted above using the refined MSP and brightness peaks in a small region (e.g., 6×6 mm) around the estimate are searched. The largest peak is the AC point. The PC point can be refined using the PC estimate above and the refined MSP. A Canny edge map of the MSP image can be computed. Again, a small region (e.g., about 6 mm×6 mm) can be searched for a point that lies on a Canny edge and for which the image gradient is most nearly parallel to the AC-PC direction. The point is moved about 1 mm along the AC-PC direction, towards PC. The largest intensity peak in the direction perpendicular to AC-PC is taken to be the PC point.

It will be appreciated that when the target is a tumor or ventricle to be infused or the like, the AC-PC points typically will not be used to provide guidance.

The Navigation-Insertion step may include further aspects as described in Table 3A:

TABLE 3A
Navigate-Insertion
Description

The application can provide a depth value to set on the
guide sheath 112 and/or catheter 150 prior to insertion.
The application can prompt with scan parameters for
oblique coronal and sagittal planes aligned to the
trajectory. Also for an oblique axial perpendicular
to the trajectory.
On receiving coronal or sagittal images, the
application can display an overlay graphic
indicating the planned trajectory. The
most recent coronal and sagittal images
can appear together in a 1 × 2 display.
On receiving a trajectory axial scan perpendicular
to the trajectory, the application can segment out
the cross-sections of the guide sheath 112 and/or
the catheter 150 to determine the actual path being
followed.
On receiving a trajectory axial scan perpendicular
to the trajectory, the application can display
two viewports containing:
the axial stack with graphic overlays showing
the detected path of the guide sheath 112
and/or catheter 150 on each image, an anatomic
axial view through the target showing the
planned target and the target projected from
the detected path of the guide sheath 112 and/or
catheter 150. An error value can show the distance
between the current projected target and the planned target.
If multiple trajectories have been defined for a single
entry, the application can display the trajectory that
is currently aligned during insertion.

The application may provide a depth value that is the expected distance from the target T to the top of the targeting cannula 60. The operator can measure the depth value distance from the distal tip 150t of the catheter 150 and/or distal end 112d of the guide sheath 112 and mark the proximal end point on the catheter 150 (e.g., with a sterile marker) and/or guide sheath 112. The depth stop 70 can then be secured at the marked location and the measured insertion distance verified. The depth stop 70 is configured to limit a distance that the catheter 150 extends into the body of a patient when the depth stop is inserted within the targeting cannula 60, so that full insertion of the catheter 150 up to the depth stop 70 will provide the desired insertion depth through the guide sheath 112.

In the event that the placement is not acceptable, the user may opt to proceed to the X-Y Adjustment workflow step as described in Table 3B:

TABLE 3B
Refine-Adjust X-Y Offset
Description

The X-Y Adjustment step can display the current
target and projected point as annotations to the image
data that was acquired during the Target Refinement step.
This step can prompt the user to acquire 2D images
with scan plane parameters such that the image lies
perpendicular to the trajectory and through the pivot point.
On receiving a 2D image through the pivot point, the step
can calculate the current projected target.
This step can display lines from the current projected
target to the revised target that indicate the track
the projected target would travel if the X and Y offset
wheels were turned independently. The lines can be
colored to match colors on the control wheels for X
and Y offset respectively. A tool-tip (e.g., pop-up) can
provide text to describe the necessary action.
(For example: “Turn X-offset knob to the Left”)
This step can display an annotation indicating the
location of the original planned target.
When drawing the target and the current projection
of the trajectory path, the annotations can be drawn to
match the physical size of the guide sheath 112 and/or
the catheter 150 diameter.

After the guide sheath 112 and catheter 150 have been placed and the position has been accepted by the user, the user may proceed to the substance delivery or withdrawal step.

Again, it is noted that functional patient data can be obtained in near real-time and provided to the circuit 30c/workstation 30 on the display 32 with the visualizations of the patient anatomy to help in refining or planning a trajectory and/or target location for placement of the guide sheath 112 and/or catheter 150.

The system 10 can provide a UI to set target points so that the trajectories through potential entry points can be investigated. The user may opt to overlay the outlines from a standard brain atlas over the patient anatomy for comparison purposes which may be provided in color with different colors for different structures. When using the brain atlas, the user may opt to show either just the target structure (STN or GPi) or all structures. In either case, a tooltip (e.g., pop-up) can help the user to identify unfamiliar structures. The user may also opt to scale and/or shift the brain atlas relative to the patient image to make a better match. To do this, the user may drag the white outline surrounding the brain atlas template. Fiber track structures and/or functional information of a patient's brain can be provided in a visually prominent manner (e.g., color coded or other visual presentation) for a surgeon's ease of reference.

The UI can display images and information that enable the user to see how well the guide sheath 112 and/or the catheter 150 is following the planned trajectory. The user may opt to scan Axial, Coronal and Sagittal slabs along the catheter 150 to visually determine the guide sheath 112 and/or catheter 150 alignment in those planes. The user can also scan perpendicular to the guide sheath 112 and/or catheter 150. In that case, the circuit 30c (e.g., software) can automatically identify where the guide sheath 112 and/or catheter 150 is in the slab and it then shows a projection of the current path onto the target plane to indicate the degree and direction of error if the current path is continued. The user can perform these scans multiple times during the insertion. The automatic segmentation of the guide sheath 112 and/or the catheter 150 and the display of the projected target on the target plane provide fully-automatic support for verifying the current path. The Coronal/Sagittal views can provide the physician with a visual confirmation of the guide sheath 112 and/or the catheter 150 path that does not depend on software segmentation.

After completing the initial insertion of the guide sheath 112 and/or the catheter 150, the user (e.g., physician) may find that either the placement does not correspond sufficiently close or perfectly to the plan, or the plan was not correct. The UI can support functionality whereby the physician can withdraw the guide sheath 112 and/or the catheter 150 and use the X and Y offset adjustments to obtain a parallel trajectory to a revised target. The UI can prompt the user or otherwise acquire an image slab through the distal end of the guide sheath 112 and/or the tip of the catheter 150. The UI can display the slab and on it the user may opt to modify the target point to a new location or accept the current position as final.

The UI can also support the user in adjusting a small X-Y offset to set the targeting cannula 60 to a trajectory parallel to the original one. The UI can provide visualization of the position of the guide sheath 112 and/or the catheter 150 tip relative to the target T and with instructions on what physical adjustments to make to obtain the desired parallel trajectory (shown as “turn Y wheel to the right”) and the projected error.

After the angular and/or X-Y adjustments are made, the guide sheath 112 and/or the catheter 150 insertion is carried out in the same manner as described above.

After the catheter 150 has been inserted and had its position verified by the physician, the UI can prompt the physician to begin delivery of the substance to the target via the catheter 150. In some embodiments, a test spray of a biocompatible fluid of similar density to the target therapeutic substance (e.g., saline) may be first delivered to the target.

The physician (or other operator) then actuates the pump (e.g., a syringe) 82 to begin driving a flow of the therapeutic substance through the tubing 84 and the transfer tube 155 of the catheter 150. A mass flow of the substance exits the catheter 150 through the exit port at the tip 150t into the target region T or the vicinity of the target region T.

Using MRI image data, the system 10 may render or generate near real time visualizations of the infused or delivered substance along with the near real time visualizations of the target anatomical space and the catheter 150 in the UI. That is, in the same or similar manner to the segmentation and visualization/display of the patient anatomy, the application can segment out the cross-sections of the delivered substance to determine the actual volume occupied by the delivered substance. Scans of scan planes proximate the distal tip 150t of the catheter 150 or associated with target regions can be acquired. The MR image data can be obtained and the actual distribution of the delivered substance in tissue can be shown on the display. These visualizations can be dynamically rendered (e.g., in near real time) to show the dynamic dispersion and/or infusion pattern and/or path of the delivered substance. In some embodiments, an MR contrast agent or fluid can be provided in the delivered substance having an increased SNR relative to the tissue.

With reference to FIGS. 5-9, an example catheter 150 according to embodiments of the present invention is shown. The catheter 150 has a long catheter body 150b with a proximal end portion 150p and a distal end portion 150d with a medial segment 150m extending therebetween. The length of the catheter body 150d can be in a range of 0.5-10 feet, typically in a range of about 3-6 feet, such as about 45 inches to about 51 inches. At least a segment of a distal end portion 150d is flexible as discussed above and can optionally be MRI compatible. The entire distal end portion 150d may be devoid of a rigid material. In other embodiments, an intermediate segment of the distal end portion 150d may have increased flexibility relative to the tip and a more proximal segment adjacent the skull. The catheter 150 may be particularly well-suited for delivering a substance into a brain of a patient. As also discussed above, the catheter 150 has a proximal end that may optionally be provided with a connector 151, shown as a luer connector. The transfer tube 155 extends through an outer tube 355. The outer tube 355 is coupled to an adapter 151a that attaches the outer tube 355 to the connector 151.

As shown, a second tube 255 extends along and inside a sub-length Li of the outer tube 355, surrounding the transfer tube 155. The second tube 255 can have a proximal end 255p that resides along the medial segment 150m of the catheter 150, a longitudinally spaced apart distance from the connector 151. The proximal end 255p of the second tube 255 can reside at a location that is at a middle or medial segment of the outer tube 355.

An internal support tube 1255 can reside at the proximal end portion 150p of the catheter 150 about a sub-length of the transfer tube 155 at a location corresponding to the adapter 151a and the connector 151.

The transfer tube 155 extends along and outside the longitudinally opposing ends of the outer tube 355 with a distal end 155d of the transfer tube defining the tip 150t of the catheter 150 defining the exit port 150e.

The transfer tube 155 can be affixed to the outer tube 355 and the second tube 255. An adhesive 257 such as LOCTITE® can reside between the transfer tube 155 and the second tube 255 and/or the second tube 255 and the outer tube 355. Different formulations of adhesive 257 such as LOCTITE® UV adhesive 3311 and LOCTITE® adhesive 4014 can be used at different locations.

The outer tube 355 can have a thicker wall thickness than the transfer tube 155 and the second tube 255. The outer tube 355 can have a tapered distal end portion 355d that tapers in the axial direction from a smaller outer diameter at the distal end portion 355d. The outer tube 355 can be flexible tubing. The outer tube 355 can be directly attached to the second tube 255. The second tube 255 can be directly attached to a sub-length of the transfer tube 155. No ceramic or rigid material is required to be used for supporting the distal end portion 150d or at least an intermediate segment of the distal end portion 150d (FIG. 9) of the catheter 150.

The catheter body 150b can have a constant/uniform wall thickness and outer diameter along the medial segment 150m upstream of the distal end 355d to the connector 151 that is defined by the outer tube 355.

The transfer tube 155 can have an inner diameter of 0.200 mm and an outer diameter of 0.360 mm. The second tube 255 can have an inner diameter of 0.450 mm and an outer diameter of 0.673 mm. The outer tube 355 can have a maximal outer diameter that is uniform over its length of 2 F-8 F.

The transfer tube 155 and the second tube 255 can be formed of fused silica.

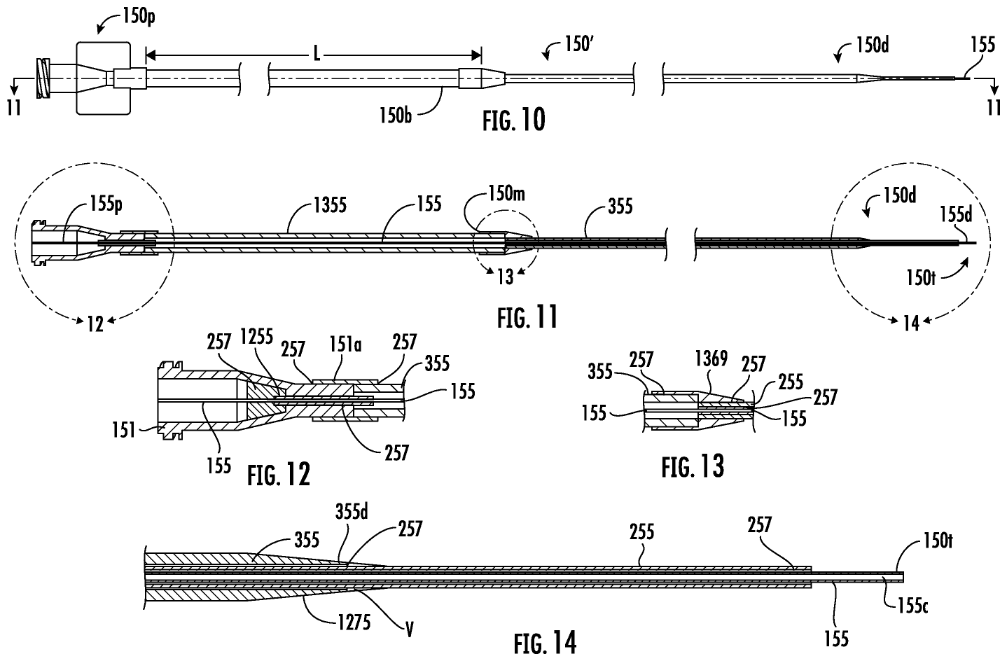

Referring now to FIGS. 10-14, another embodiment of a catheter 150′ is shown. The catheter 150′ is similar to that discussed with respect to FIGS. 5-9. In this embodiment, a conformal outer sleeve 1275 can extend over at least a portion of the outer tube 355 and the second tube 255 at the distal end 150d of the catheter 150′.

Also, the outer tube 355 can be provided as two cooperating segments a first outer tube 355 that merges into a second outer tube 1355 at a medial segment 150m of the catheter body 150b. The second outer tube 1355 can have a length in a range of 0.5-5 feet, such as about 3 feet, in some embodiments. An adapter 1360 can be used to couple the two outer tubes 355, 1355. The adapter 1360 can taper axially from a smaller outer diameter to a larger outer diameter, or from a larger to a smaller outer diameter, in an axial direction toward or away from the proximal end, optionally with a connector 151. The second outer tube 1355 that resides closer to the connector 151 can have a greater or lesser wall thickness and greater or lesser outer diameter than the first outer tube 355.

The conformal outer sleeve is polymeric that surrounds and fits tightly about the distal end portion 355d of the outer tube 355 and the exposed segment of the second tube 255.

An annular void V (FIG. 14) is defined between the outer distal end surface of the outer tube 355 and the outer wall of the second tube 255. The annular void V can be at least partially filled with a rigid or semi-rigid or even flexible adhesive 257 to form a solid or semi-solid ramp. In this way, the step is effectively smoothed of eliminated on the outer diameter of the catheter thereat.

According to some embodiments, the conformal polymeric sleeve 1275 is formed of polyethylene terephthalate (PET). According to some embodiments, the conformal polymeric sleeve 1275 is an elastomeric shrinkable sleeve.

According to some embodiments, the inner diameter of the transfer tube 155 is in the range of from about 10 μm to 1 mm and, in some embodiments, is about 200 μm. According to some embodiments, the outer diameter of the transfer tube 155 is in the range of from about 75 μm to 1.08 mm and, in some embodiments is about 360 μm. According to some embodiments, the length of the exposed section of the transfer tube 155 at the tip 150t of the catheter 150 is in the range of from about 1 mm to 50 mm and, in some embodiments is about 3 mm.

According to some embodiments, the inner diameter of the second tube 255 is in the range of from about 85 μm to 1.1 mm and, in some embodiments, is about 450 μm. According to some embodiments, the outer diameter of the second tube 255 is in the range of from about 150 μm to 1.5 mm and, in some embodiments, is about 673 μm. According to some embodiments, the length of the exposed section of the second tube 255 (i.e., the section of the second tube 255 extending distally beyond the outer tube 355) is in the range of from about 1 mm to 75 mm and, in some embodiments is about 15 mm.

According to some embodiments, the inner diameter of the second tube 255 is in the range of from about 160 μm to 1.55 mm and, in some embodiments, is about 750 μm. According to some embodiments, the outer diameter of the uniform diameter section 355u of the outer tube 355 is in the range of from about 500 μm to 4 mm and, in some embodiments, is about 1.6 mm.

According to some embodiments, the overall length of the second tube 255 is in the range of from about 0.5 inch to 20 inches and, in some embodiments, is in the range of from about 10 to 14 inches. According to some embodiments, the length of the tapered section of the outer tube 355 is in the range of from about 6 to 9 mm.

According to some embodiments, the thickness of the conformal polymeric sleeve 1275 is in the range of from about 40 to 60 μm.

As best seen in FIG. 9, the catheter 150 can be a stepped catheter with three co-axially disposed step segments (the outer surfaces of the transfer tube 155, the second tube 255 and the outer tube 355 or conformal polymeric sleeve 1275, respectively) having different outer diameters and separated by the steps or rises. The step formed by the end face 255B and/or end face 355B is a sharp step.

The catheter 150, 150′ may be a unitary, integral structure having no relatively slidably elements.

The conformal polymeric sleeve 1275 (FIG. 14) may beneficially provide a lubricious surface to reduce shear force on the brain or other tissue during insertion.

The step at the end face(s) 255B, 355B can serve to reduce or prevent reflux of the delivered substance. The provision of an exposed section of the transfer tube 155 having the aforedescribed length and inner diameter has also been found to provide beneficial reflux resistance performance.

According to some embodiments, the delivered substance is delivered to a patient's brain through the exit opening at the tip 150t of the catheter 150 at a delivery rate in the range of from about 1 to 3 μL/minute.

The catheter 150, 150′ may be particularly well-suited for delivering therapies, optionally comprising stem cells, to the brain tissue of a patient. According to method embodiments of the invention, stem cells are delivered or injected through the catheter into a patient as described herein. The stem cells may be suspended in a liquid composition or suspension that is delivered through the catheter 150, 150′.

As discussed herein, insertion of the surgical catheter 150, 150′ can be tracked in near real time by reference to a void in the patient tissue caused by the catheter 150, 150′ and reflected in the MR image. In some embodiments, one or more MM-visible fiducial markers may be provided on the surgical catheter 150, MR scanned and processed, and displayed on the UI. In some embodiments, the surgical catheter 150, 150′ may itself be formed of an MM-visible material, MR scanned and processed, and displayed on the UI.

While the surgical catheters 150, 150′ have been identified herein for delivering a substance to a patient, in accordance with some embodiments of the invention, the surgical catheters and methods can be used to withdraw a substance (e.g., spinal fluid) from a patient. Thus, it will be appreciated that surgical catheters and methods as disclosed herein can be used to transfer a substance into and/or from a patient.

While the surgical catheters have been described herein primarily with reference to MM-guided insertion and infusion procedures, in some embodiments the catheters can be used in procedures without MRI guidance, such as CT-guided systems and may use other materials than described above.

The systems, catheters, methods and procedures described herein may likewise be used as an acute or chronic delivery catheter. For example, the delivery catheters 150, 150′ may be installed in a patient for chronic delivery as described in PCT Published Patent Application No. WO 2011/130107 A2 (Attorney Docket No. 9450-75WO), the contents of which are hereby incorporated by reference as if recited in full herein.

According to some embodiments, the substance delivered via the delivery catheter includes radioactive objects such as radioactive seeds. In this event, the delivery catheter may include a suitable radiation shield or shielding material in order to reduce or prevent the exposure of tissue outside the target region to radiation from the radioactive objects.

The system 10 may also include a decoupling/tuning circuit that allows the system to cooperate with an MM scanner 20 and filters and the like. See, e.g., U.S. Pat. Nos. 6,701,176; 6,904,307 and U.S. Patent Application Publication No. 2003/0050557, the contents of which are hereby incorporated by reference as if recited in full herein.

The system 10 can include circuits and/modules that can comprise computer program code used to automatically or semi-automatically carry out operations to generate visualizations and provide output to a user to facilitate MRI-guided diagnostic and therapy procedures.

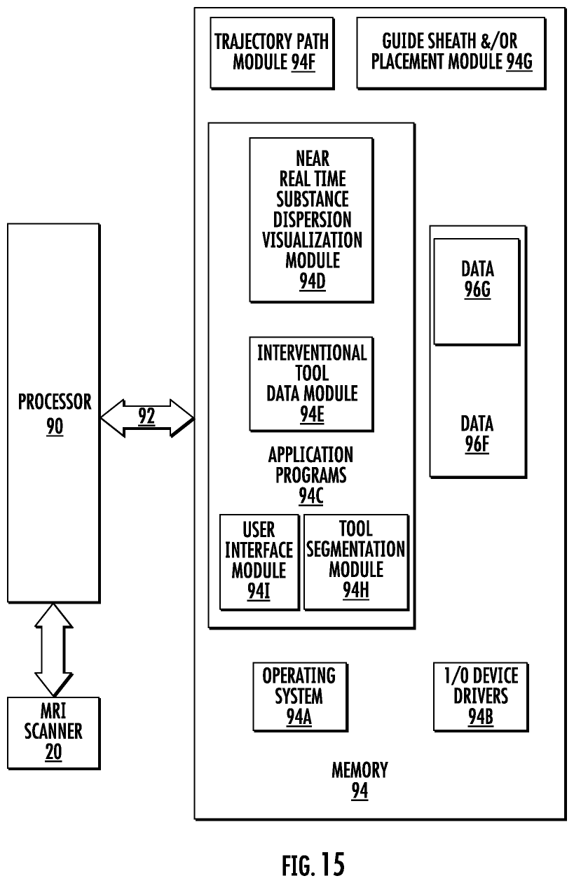

FIG. 15 is a schematic illustration of a circuit or data processing system that can be used with the system 10. The circuits and/or data processing systems may be incorporated in one or more digital signal processors in any suitable device or devices. The processor 90 communicates with an MRI scanner 20 and with memory 94 via an address/data bus 92. The processor 90 can be any commercially available or custom microprocessor. The memory 94 is representative of the overall hierarchy of memory devices containing the software and data used to implement the functionality of the data processing system. The memory 94 can include, but is not limited to, the following types of devices: cache, ROM, PROM, EPROM, EEPROM, flash memory, SRAM, and DRAM.

The memory 94 may include several categories of software and data used in the data processing system: the operating system 94A; the application programs 94C; the input/output (I/O) device drivers 94B; and data 96F. The data 96F can also include predefined characteristics of different surgical tools and patient image data 94G. The application programs 94C can include a Near Real-Time Substance Dispersion Visualization Module 94D, Interventional Tool Data Module 94E, a Tool Segmentation Module 94H (such as segmentation modules for a targeting cannula, a trajectory guide frame and/or base, and a delivery catheter), and a workflow group User Interface Module 94I (that facilitates user actions and provides guidance to obtain a desired trajectory or a desired drug dispersion pattern, such as physical adjustments to achieve same), a Trajectory Path Selection Module 94F, and a Guide Sheath and/or Catheter Placement Module 94G.

As will be appreciated by those of skill in the art, the operating systems 94A may be any operating system suitable for use with a data processing system, such as OS/2, AIX, DOS, OS/390 or System390 from International Business Machines Corporation, Armonk, NY, Windows CE, Windows NT, Windows95, Windows98, Windows2000 or other Windows versions from Microsoft Corporation, Redmond, WA, Unix or Linux or FreeB SD, Palm OS from Palm, Inc., Mac OS from Apple Computer, LabView, or proprietary operating systems. The I/O device drivers 94B typically include software routines accessed through the operating system 94A by the application programs 94C to communicate with devices such as I/O data port(s), data storage 96F and certain memory 94 components. The application programs 94C are illustrative of the programs that implement the various features of the data processing system and can include at least one application, which supports operations according to embodiments of the present invention. Finally, the data 96F represents the static and dynamic data used by the application programs 94C, the operating system 94A, the I/O device drivers 94B, and other software programs that may reside in the memory 94.

While the present invention is illustrated, for example, with reference to the Modules 94C, 94D, 94E, 94H, 94I, 94F, 94G being application programs in FIG. 15, as will be appreciated by those of skill in the art, other configurations may also be utilized while still benefiting from the teachings of the present invention. For example, one or more of the Modules may be incorporated into the operating system 94A, the I/O device drivers 94B or other such logical division of the data processing system. Thus, the present invention should not be construed as limited to the configuration of FIG. 15, which is intended to encompass any configuration capable of carrying out the operations described herein. Further, one or more of Modules can communicate with or be incorporated totally or partially in other components, such as a workstation, an MM scanner, an interface device. Typically, the workstation 30 will include the Modules and the MR scanner with include a module that communicates with the workstation 30 and can push image data thereto.

The I/O data port can be used to transfer information between the data processing system, the circuit 30c or workstation 30, the MRI scanner 20, and another computer system or a network (e.g., the Internet) or to other devices controlled by or in communication with the processor. These components may be conventional components such as those used in many conventional data processing systems, which may be configured in accordance with the present invention to operate as described herein.

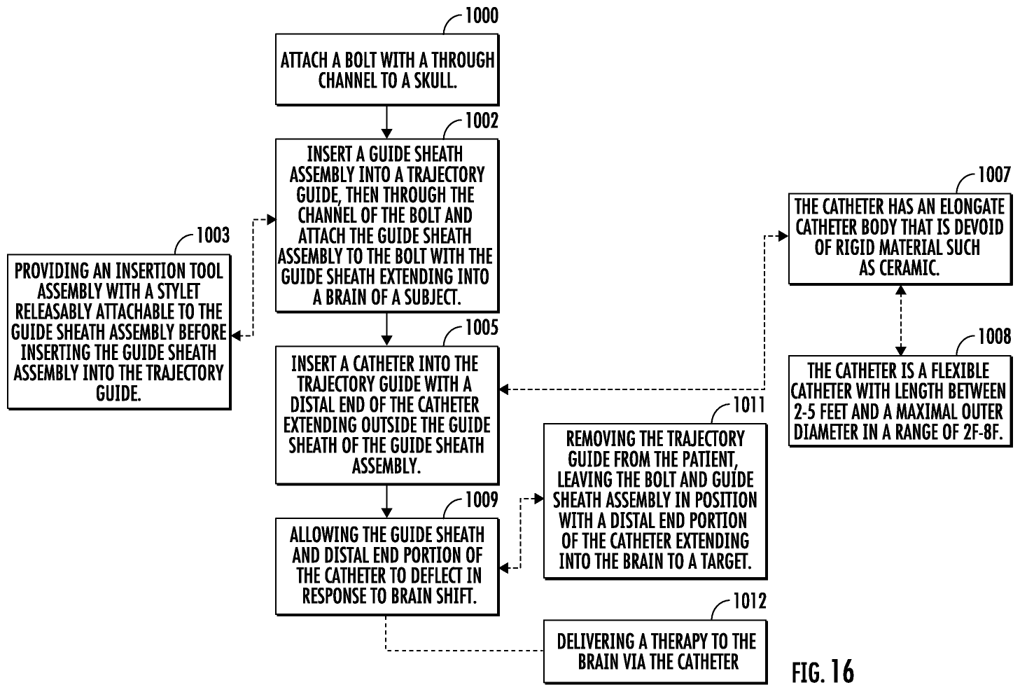

FIG. 16 illustrates example actions that can be used to carry out a therapy on a patient. As shown, a bolt with a through channel is attached to a skull (block 1000). A guide sheath assembly is inserted into a trajectory guide, then into/through the channel of the bolt and the guide sheath assembly is attached to the bolt with the guide sheath extending into the brain of a subject (block 1002). A catheter is inserted into the trajectory guide with a distal end of the catheter extending outside the guide sheath of the guide sheath assembly (block 1005). Allowing the guide sheath and distal end portion of the catheter to deflect in response to brain shift while retaining the trajectory to target (block 1009).

An insertion tool assembly with a stylet can be provided. Before inserting the guide sheath assembly into the trajectory guide, the insertion tool assembly is releasably attached to the guide sheath assembly (block 1003). The insertion tool assembly is removed before inserting the catheter into the trajectory guide.

The catheter has an elongate body that is devoid of rigid material such as ceramic (block 1007). The catheter is a catheter with a length in a range of 0.5-5 feet and a maximal outer diameter in a range of 2F-8F (block 1008).

The trajectory guide can be removed from the subject/patient leaving the bolt and guide sheath assembly in position with a distal end portion of the catheter extending into the brain to a target (block 1012).

It is noted that any one or more aspects or features described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

Other systems, methods, and/or computer program products according to embodiments of the invention will be or become apparent to one with skill in the art upon review of the following drawings and detailed description. It is intended that all such additional systems, methods, and/or computer program products be included within this description, be within the scope of the present invention, and be protected by the accompanying claims.

In the drawings and specification, there have been disclosed embodiments of the invention and, although specific terms are employed, they are used in a generic and descriptive sense only and not for purposes of limitation, the scope of the invention being set forth in the following claims. Thus, the foregoing is illustrative of the present invention and is not to be construed as limiting thereof. More particularly, the workflow steps may be carried out in a different manner, in a different order and/or with other workflow steps or may omit some or replace some workflow steps with other steps. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed:

1. An intrabrain catheter, comprising:
an elongate body having a length in a range of 0.5-10 feet, the elongate body comprising a transfer tube that extends a full length of the elongate body and that extends out a distal end portion thereof to define an exposed tip, wherein the intrabrain catheter has a proximal end portion that is configured to be external to a patient, wherein the proximal end portion has sufficient flexibility to be able to bend at least 30 degrees relative to an axially extending straight linear axis in an unloaded, normal orientation, wherein the intrabrain catheter has a distal end portion with sufficient rigidity to maintain a straight linear orientation for insertion through a tubular guide of a trajectory frame, and wherein the distal end portion of the intrabrain catheter is configured to have sufficient flexibility to be able to deflect in response to a deflection force applied by brain tissue during brain shift associated with patient movement.

2. The intrabrain catheter of claim 1, wherein the elongate body comprises an outer tube that is polymeric and that surrounds a length of the transfer tube, wherein the outer tube has a wall thickness that is greater than the transfer tube, and wherein the transfer tube is indirectly coupled to the outer tube and is non-extendable relative to the outer tube.

3. The intrabrain catheter of claim 2, wherein a distal end portion of the outer tube tapers radially outward in an axial direction from the transfer tube to an outer diameter, wherein the outer tube has a length that is less than a length of the elongate body and is in a range of 6-36 inches, and wherein the transfer tube has a length that is longer than the outer tube.

4. The intrabrain catheter of claim 3, further comprising a second tube that is attached to the transfer tube and that resides between the transfer tube and the outer tube, wherein the second tube is formed of the same material as the transfer tube and terminates a distance outside the outer tube before the tip of the intrabrain catheter.

5. The intrabrain catheter of claim 4, wherein the second tube and the transfer tube are both formed of fused silica.

6. The intrabrain catheter of claim 1, wherein at least a segment of the distal end portion of the elongate body of the intrabrain catheter is devoid of a support tube of rigid material such as ceramic.

7. The intrabrain catheter of claim 1, wherein the intrabrain catheter is MRI-compatible.

8. The intrabrain catheter of claim 1, wherein the proximal end portion of the elongate body is coupled to a connector with an internal cavity surrounding an exposed sub-length of the transfer tube.

9. The intrabrain catheter of claim 1, wherein the elongate body comprises a first polymeric outer tube coupled to a second polymeric outer tube via an adapter member, with the first polymeric outer tube extending longitudinally spaced apart from the second polymeric outer tube, and wherein the first polymeric outer tube resides closer to the proximal end portion than the second polymeric outer tube, optionally wherein the first polymeric outer tube has a greater outer diameter and wall thickness than the second polymeric outer tube.

10. The intrabrain catheter of claim 1, wherein the elongate body is provided by a polymeric outer tube with a constant outer diameter extending between a proximal end to a segment merging into a tapered distal end segment.

11. The intrabrain catheter of claim 1, wherein the elongate body comprises a polymeric outer tube that is directly attached to a second tube extending about the transfer tube along a sub-length of the elongate body, and wherein the second tube is directly attached to the transfer tube.

12. A medical system, comprising:
an intrabrain catheter;
a sheath assembly comprising a guide sheath with a proximal end and an opposing distal end and with a lumen extending therethrough, wherein the proximal end comprises a shoulder that extends radially outward from the lumen;
a bolt configured to threadably engage a skull of a patient, wherein the bolt comprises an open channel that extends axially therethrough, wherein the guide sheath is configured to reside in the open channel of the bolt with the distal end residing distally of the bolt, and wherein the intrabrain catheter is configured to reside in the guide sheath with a distal end thereof residing external to the guide sheath;
a seal member inside the bolt adjacent the shoulder of the guide sheath; and
a bolt nut configured to couple to the bolt.

13. The medical system of claim 12, wherein the proximal end of the sheath assembly terminates inside the bolt.

14. The medical system of claim 12, wherein the bolt nut has a distal portion that is configured to apply a clamping force against the seal member.

15. The medical system of claim 12, wherein the seal member comprises an O-ring, optionally a silicone O-ring.

16. The medical system of claim 12, wherein the intrabrain catheter comprises an elongate body having a length in a range of 0.5-5 feet, the elongate body comprising a transfer tube that extends a full length of the elongate body and that extends out a distal end portion thereof to define an exposed tip, wherein the intrabrain catheter has a proximal end portion that is configured to be external to a patient, wherein the proximal end portion has sufficient flexibility to be able to bend at least 30 degrees relative to an axially extending straight linear axis in an unloaded, normal orientation, wherein the intrabrain catheter has a distal end portion with sufficient rigidity to maintain a straight linear orientation for insertion through a tubular guide of a trajectory frame, and wherein the distal end portion of the intrabrain catheter is configured to have sufficient flexibility to be able to deflect in concert with the guide sheath response to a deflection force applied by brain tissue during brain shift associated with patient movement.

17. The medical system of claim 12, wherein the guide sheath is provided as a plurality of guide sheaths, each guide sheath in a different length to thereby allow a user to select an appropriate guide sheath to use for a particular medical procedure.

18. A method of providing a therapy to a brain of a subject, comprising:
attaching a bolt with a through channel to a skull of the subject;
inserting a guide sheath assembly into a trajectory guide, then into the through channel of the bolt;
attaching the guide sheath assembly to the bolt with a guide sheath of the guide sheath assembly extending distally out of the through channel of the bolt into a brain of the subject;
inserting a catheter into the trajectory guide with a distal end of the catheter extending outside of the guide sheath; and
allowing the guide sheath and distal end portion of the catheter to deflect in response to brain shift.

19. The method of claim 18, further comprising providing an insertion tool assembly with a stylet releasably attached to the guide sheath assembly and before inserting the guide sheath assembly into the trajectory guide, slidably forcing the guide sheath assembly to couple to the bolt using the insertion tool assembly, then removing the insertion tool assembly before inserting the catheter into the trajectory guide.

20. The method of claim 18, further comprising delivering a therapy to the brain using the catheter.

21. The method of claim 18, wherein the catheter has an elongate catheter body that is devoid of ceramic and has a length in a range of 0.5-5 feet and a maximum outer diameter in a range of 2 F-8 F.

22. The method of claim 18, the method further comprising providing a plurality of guide sheath assemblies in different lengths and allowing a user to select one for the inserting step.
